(19) United States
(12) Patent Application Publication
(10) Pub. No.: US 2024/0255521 A1
(43) Pub. Date: Aug. 1, 2024

(54) Cilia Protein as Biomarkers and Methods of Use

(71) Applicants: The Medical College of Wisconsin, Inc., Milwaukee, WI (US); Duke University, Durham, NC (US)

(72) Inventors: Ramani Ramchandran, Pewaukee, WI (US); Kevin Rarick, Milwaukee, WI (US); Rahima Zennadi, Durnham, NC (US)

(21) Appl. No.: 18/561,185

(22) PCT Filed: May 20, 2022
(86) PCT No.: PCT/US2022/030298

§ 371 Date: Nov. 15, 2023

Related U.S. Application Data
(60) Provisional application No. 63/191,126, filed on May 20, 2021.

(51) Int. Cl. G01N 33/68; G01N 33/543; G01N 33/58; G01N 33/80

(52) U.S. Cl. CPC G01N 33/6854; G01N 33/543; G01N 33/58; G01N 33/80; G01N 2333/914; G01N 2800/22; G01N 2800/2871; G01N 2800/321; G01N 2800/347; G01N 2800/50

(57) ABSTRACT

The present invention provides methods and kits for detecting cilium markers in samples and uses thereof for detecting and treating endothelial damage or dysfunction or vascular injury in the subject. In one aspect, the disclosure provides a method of detecting endothelial damage or dysfunction or vascular injury in a subject in need thereof, the method comprising: detecting one or more markers of cilium in a biological sample from the subject, wherein a higher level of cilium detected in the biological sample compared to control indicates endothelial damage or dysfunction or vascular injury.

### CROSS-REFERENCE TO RELATED APPLICATIONS

This application claims priority to U.S. Provisional Application No. 63/191,126 filed on May 20, 2021, the contents of which are incorporated by reference in their entireties.

### STATEMENT REGARDING FEDERALLY SPONSORED RESEARCH

This invention was made with government support under grant number R61HL154254 awarded by the National Institutes of Health. The government has certain rights in this invention.

### SEQUENCE LISTING

A Sequence Listing accompanies this application and is submitted as an ASCII text file of the sequence listing named “650053.00884_ST25.txt” which is 1518 bytes in size and was created on May 19, 2022. The sequence listing is electronically submitted via EFS-Web with the application and is incorporated herein by reference in its entirety.

### BACKGROUND

Cilia, microtubule-based organelles that project from the apical luminal surface of endothelial cells (ECs), are widely regarded as low-flow sensors. Previous reports suggest that upon high shear stress, cilia on the EC surface are lost, and more recent evidence suggests that deciliation—the physical removal of cilia from the cell surface—is a predominant mechanism for cilia loss in mammalian cells.

There are need for new markers and methods of detecting changes that are caused by high or low vascular flow, leading to endothelial damage or dysfunction or vascular injury.

### SUMMARY

Cilia and its' associated proteins expressed on endothelial cells are lost upon encountering turbulent blood flow, which leads to removal of cilia from cells, and its detection on red blood cells and body fluids. This free cilia can thus be used as a biomarker for altered flow in the blood, that is associated with vascular dysfunction and injury.

In one aspect, the disclosure provides a method of detecting endothelial damage or dysfunction or vascular injury in a subject in need thereof, the method comprising: detecting one or more markers of cilium in a biological sample from the subject, wherein a higher level of cilium detected in the biological sample compared to control indicates endothelial damage or dysfunction or vascular injury.

In another aspect, the disclosure provides a kit comprising: at least one antibody that binds to at least one cilium marker, and instructions for use. The kit may further comprise at least one secondary agent that has a detectable label. The one or more antibodies may bind to a cilium marker selected from the group consisting of ARL13b, γ-tubulin, IFT88, Inversin, nuclear factor-erythroid factor 2-related factor 2 (NRF2), and acetylated-α-tubulin.

In another aspect, the disclosure provides a method of detecting an occlusive event associated with sickle cell disease (SCD) in a subject having SCD, the method comprising: detecting one or more markers of cilium in a first biological sample from the subject.

In a further aspect, the disclosure provides a method of detecting cilia on red blood cells in a sample from a subject, the method comprising obtaining a blood sample from a subject; isolating the red blood cells (RBCs) from the sample, and detecting one or more markers of cilium on the surface of the red blood cells.

### BRIEF DESCRIPTION OF THE DRAWINGS

FIG. 1. Shear stress causes brain ECs to express fewer cilia proteins in vitro. HBMVECs were subjected to graded strengths of shear stress (2 dyne/cm², 4 dyne/cm², and 10 dyne/cm²) by the Ibidi flow system. A total of 2 dyne/cm² was utilized as a “steady-state” flow condition. Following flow with durations as indicated, the expression of cilia-associated proteins was quantified as MFI by flow cytometry. NRF2, the transcription factor reported to control cilia formation and function, was also included in the study. Expression of proteins in the samples was normalized against their respective “no flow” controls. ANOVA was performed to compare between the experimental groups versus steady-state control group for 10-minute or 24-hour time points. ANOVAs were 2 way. Analysis was also performed between 10 minutes and 24 hours in 2 dyne/cm² group. In all 3 protein expressions, no statistical difference was observed between 4 dyne/cm² and 10 dyne/cm² groups. *P<0.05, **P<0.01, ***P<0.001, ****P<0.0001. For all groups reported in this figure, n=6 except for IFT88 (n=5 for 10 dyne group).

FIG. 2A-2D. Shear stress causes deciliation of endothelial and epithelial cells in vitro. (A-D) HUVECs grown on monolayer. Fluid shear stress of 20 dyne/cm² was perfused onto the cells, and the perfusate was collected. The drop concentrated perfusate was analyzed with DIC microscopy and stained with markers for cilia (acetylated-α-tubulin) (B) and basal body (γ-tubulin) (C). Enlarged images are also shown in the boxes. (D) Merged image is shown. (E and F) Immunostaining (acetylated-α-tubulin, green, cilia; and DAPI, blue, nucleus) for the presence and absence of the cilia from the epithelial cell population before (upper panel) and after (lower panel) the application of 10 dyne/cm² shear stress, respectively. (G and H) Phase contrast DIC image of a primary cilium in a single live cell (white dotted box). The same cell was imaged before and after 4-minute application of 10 dyne/cm² fluid shear stress. (I) Perfusate under DIC microscopy. (J) A separate experiment, where perfusate was collected and stained with ciliary marker (acetylated-α-tubulin; green) to confirm the presence of cilia using both fluorescence and phase contrast imaging (I). (K) Cilia and cell lysates were immunoblotted with cilia marker (acetylated-α-tubulin) to molecularly confirm the presence of the cilia in the perfusate (n=6). E-K represent porcine kidney epithelial cells (LLC-PK1).

FIG. 3A-3D. Shear stress results in ECs with fewer cilia proteins in vivo. Flk1^{mcherry} Arl13b^{GFP} double-transgenic zebrafish embryos at 29.5 hpf were subjected to 32° C. temperature for 3 hours. Single cells were harvested from dechorionated embryos, and expression of cilia-specific proteins was quantified by flow cytometry in live ECs (mCherry⁺) versus non-ECs (mCherry⁻). Representative dot plots show the gating strategy as applied during FACS analysis to identify ECs (A). Stress-responsive protein Klf4 was quantified in ECs (B). Protein quantification was done by measuring MFI. Cilia-specific proteins were quantified in ECs (C) as well as non-ECs (D). Arl13b expression is marked by enhanced green fluorescent protein expression. For Arl13b n=6 (for EC and non-EC); γ-tubulin n=5 (for EC and non-EC); Ift88 n=4 (EC) and n=5 (non-EC); Inversin (n=3 for EC and non-EC); Klf4 n=3 (for EC and non-EC). A linear mixed model was used to examine the differences between treatment group and control group within EC (mCherry⁺) or non-EC (mCherry⁻). Time processed nested within day was treated as random. ARL13b and Inversin expression data were log-transformed to improve fit.

FIG. 4A-4F. Sickle RBCs adhere to brain ECs triggering deciliation, and cilia are found on sickle RBCs and plasma from SCD. HBMVECs exposed to sickle (SS) or healthy (AA) RBCs were subjected to shear stress (1 dyne/cm²), and fraction of SS (n=6) and AA (n=5) RBCs adhered to ECs after stress induction was calculated, P=0.0081 (A). SS and AA RBCs were tested for ARL13b cilia prior to flow and proportion of ARL13b cilia adhered to circulating SS RBCs (n=16) versus AA RBCs (n=12) were quantified, P<0.0001 (B). After flow, ARL13b expression on SS RBCs (n=11), but not AA RBCs (n=6), upon interaction with ECs, P=0.0006 (C). (A-C) Mann-Whitney-Wilcoxon test P values are provided. Representative field (magnification 63×; scale bar=20 μm) of a smear of SS RBCs shows cilia presence on these sickle cells, detected with FITC-conjugated anti-Arl13b antibody (D). Western blot plot shows the detection of cilia-specific proteins in plasma samples of healthy controls (AA) versus sickle (SS). Red asterisk represents the top IFT88 band that was used for quantification (E). Please note that Western blots from only 4 AA and SS samples are shown in E. A separate gel for the other 6 samples was run and quantified. Quantification includes all 10 samples from each group. Cilia-specific proteins were quantified from plasma samples of healthy controls (AA) (n=10) versus sickle (SS) (n=10) and normalized against housekeeping protein bACTIN (F). *P<0.05, ***P<0.001. A 2-tailed t test or Mann-Whitney-Wilcoxon test was performed to compare between groups.

FIG. 5A-5B. Cilia shedding triggered by sickle RBCs is dependent on RBC-induced increased EC ROS generation. Human brain microvascular ECs were sham treated or treated with the NOX inhibitor apocynin (A), prior to exposure or not with sickle RBCs (SS RBCs) (n=6). Exposure of HBMVECs to SS RBCs increased ROS generation in ECs, which is dependent on NOX. Flow cytometry analysis shows ARL13b bound to SS RBCs (n=6) prior to (baseline) and after interaction with shear-stressed ECs pretreated with apocynin (B). *P<0.05, and ****P<0.0001.+^{a}=ECs treated with apocynin. For A, 1-way ANOVA test was performed, and for B, Wilcoxon's signed-rank test was performed.

FIG. 6A-6E. Attenuating ROS production rescues cilia proteins in ECs. HBMVECs were treated with ROS-inducing PMA in presence or absence of NOX inhibitor VAS2870. Untreated groups were also included as controls. ROS production was quantified as MFI by flow cytometry (A). Oxidative stress counteracting protein heme oxygenase 1 (HO-1) was quantified (B). NRF2, the transcription factor reported to control cilia formation and function, was also included in the study (C). Cilia proteins are downregulated by PMA and rescued by NOX inhibitor VAS2870 (D and E). ****P<0.0001. ANOVA (1 way) was performed, and Bonferroni's correction was used to adjust for multiple comparisons (n=5 for all groups).

FIG. 7A-7B. Validation of shear stress as induced by Ibidi flow system. Human brain microvascular endothelial cells were subjected to 10 dyne/cm2 shear stress for 24 hours, as induced by ibidi flow system and subsequently monitored for flow responsive genes KLF4 or KLF2 by qRT-PCR (A) as well as the respective proteins by flow cytometry (B). KLF4 and KLF2 gene expressions were normalized against GAPDH and plotted as fold change. *P<0.05

FIG. 8A-8F. Loss of cilia proteins from brain endothelial cells following shear stress induced by shaker method in vitro. Human brain microvascular endothelial cells were subjected to graded strengths of shear stress (4 dyne/cm2 and 10 dyne/cm2) as induced by ‘shaker’ method and subsequently the expression of cilia-specific proteins was quantified by flow cytometry. Stress-responsive proteins were quantified (A) along with non-cilia house keeping (B) or cilia-specific proteins (C-F) by measuring median fluorescent intensity. Comparison across groups (4 dyne vs. Control, 10 dyne vs. Control and 4 dyne vs. 10 dyne) were performed for all proteins. In all three group comparisons, P,0.001 for Arl13b, Tubulin, IFT88, and Inversin proteins. For Alk1, P=0.0003 for 4 dyne vs. Control, P,0.001 for 10 dyne vs. Control and P=0.0004 for 4 dyne vs. 10 dyne comparison. For Dynein, P,0.001 for 4 dyne vs. Control and 10 dyne vs. Control, and P=0.0173 for 4 dyne vs. 10 dyne comparison. N=3 for all protein targets except for β-actin, which is n=4.

FIG. 9A-9B. Scheme for shear stress EC FACS experiments in vitro and in vivo. A shows the scheme the treatment of HBMVECs post shear stress for FACS analysis. B shows the scheme for fish treatment conditions, and subsequent analysis prior to FACS.

FIG. 10. Temperature-induced shear stress experimental design in zebrafish. The 3 groups and the conditions for incubation for each are depicted in a pictorial format. All blood flow parameters were evaluated at 48 hours post fertilization (hpf) stage.

FIG. 11A-11B. PMBC flow parameters assessed at two temperatures of 32° C. and 35° C. A shows pulse, blood flow velocity, vessel diameter and shear stress measured in primordial midbrain channels (PMBCs) across the three groups (G1-G3 shown in figure S2) at 32° C. B shows pulse, blood flow velocity, vessel diameter and shear stress measured in PMBCs across the three groups (G1-G3 shown in figure S2) at 35° C. *P<0.001.

FIG. 12A-12B. DA flow parameters assessed at two temperatures of 32° C. and 35° C. A shows pulse, blood flow velocity, vessel diameter and shear stress measured in dorsal aorta (DA) across the three groups (G1-G3 shown in figure S2) at 32° C. B shows pulse, blood flow velocity, vessel diameter and shear stress measured in DA across the three groups (G1-G3 shown in figure S2) at 35° C. *P<0.001.

FIG. 13A-13B. PMBCs and DA flow parameters assessed at two temperatures of 32° C. A shows pulse, blood flow velocity, vessel diameter and shear stress measured in primordial midbrain channels (PMBCs) across the three groups (G1-G3 shown in figure S2) at 32° C. B shows pulse, blood flow velocity, vessel diameter and shear stress measured in DA across the three groups (G1-G3 shown in figure S2). *P<0.0001.

FIG. 14A-14F. Whole mount images of casper fish at 35° C. and 32° C. A,D (group 1), B,E (group 2) and C,F (group 3) Casper transparent 48 hours post fertilization (48 hpf) fish embryos incubated at 35° C. and 32° C. were stained with O-dianisidine stain (red blood cells). Note group 3 (C) embryos at 35° C. display curved axis. Anterior is left and posterior is right. Dorsal is up.

FIG. 15A-15C. Cilia on circulating mouse sickle RBCs. A and B shows blood smears from SS sickle mouse stained for IFT88 and Arl13b antibodies. The green stain is cilia on RBCs. Panel C quantification is from a different experiment from panels A & B. For the quantification of Arl13b positive cilia adhering to control (AA) vs. sickle RBCs (SS), four mice per group were used. 1 smear from each mice, and four fields in each smear were counted. Thus, a total of data from 16 smears counted from control mice and sickle mice group (n=4 per group) are presented. #P<0.0001.

### DETAILED DESCRIPTION

The present invention provides methods and kits for detecting cilia in a biological sample and uses thereof. In the present disclosure, we demonstrate that cilia serve as a biomarker for altered flow (high or low) in disease conditions, and thus may be used as a prognostic or diagnostic marker for illness in patients. Flow rate is either lower or higher in the body which can result in vascular damage and injury. Thus, ECs that underlie all blood vessels in the body are the first line of flow sensors, which reacts to the altered flow patterns by either dispensing or keeping cilium on the cell surface. Thus, the net result is more or lesser cilia in the biological fluid, which can be monitored by assays (e.g., ELISA analysis). In some embodiments, the basal body associated with cilium serve as a marker for the origin of cilium in biofluids (serum, blood, urine, cerebrospinal fluid, seminal fluid, saliva, tears, synovial fluid, breast milk, bile, amniotic fluid, aqueous humor, vaginal lubrication, sweat, lymph, bone marrow).

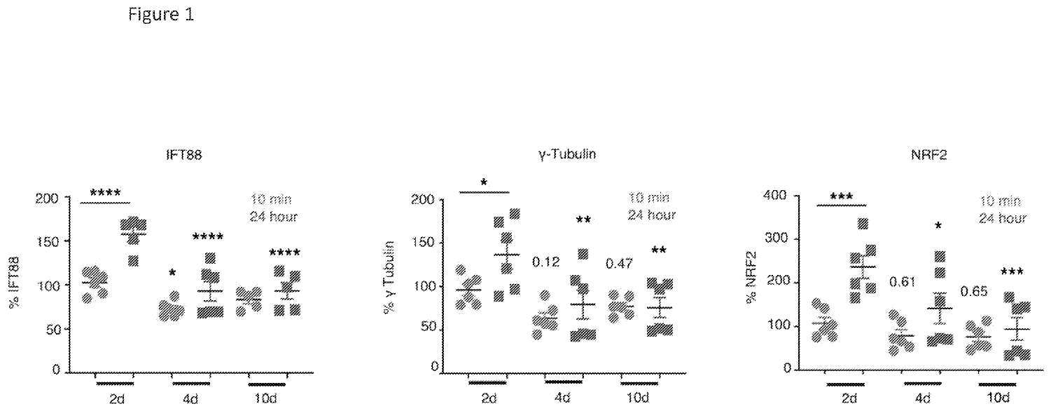

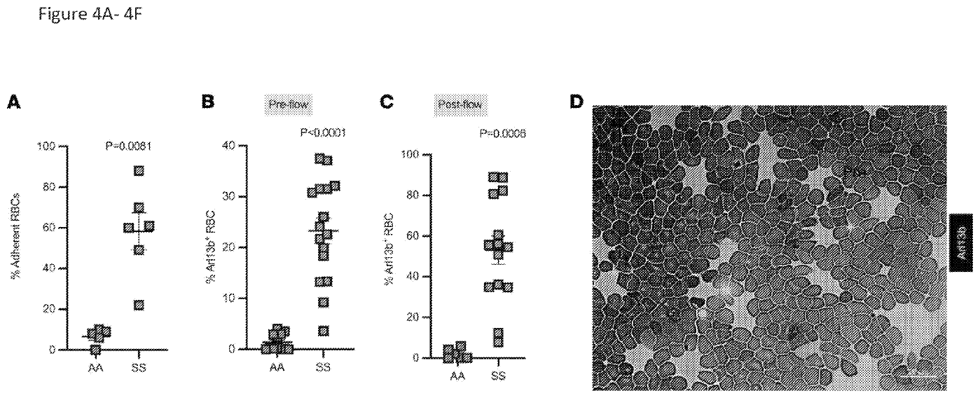

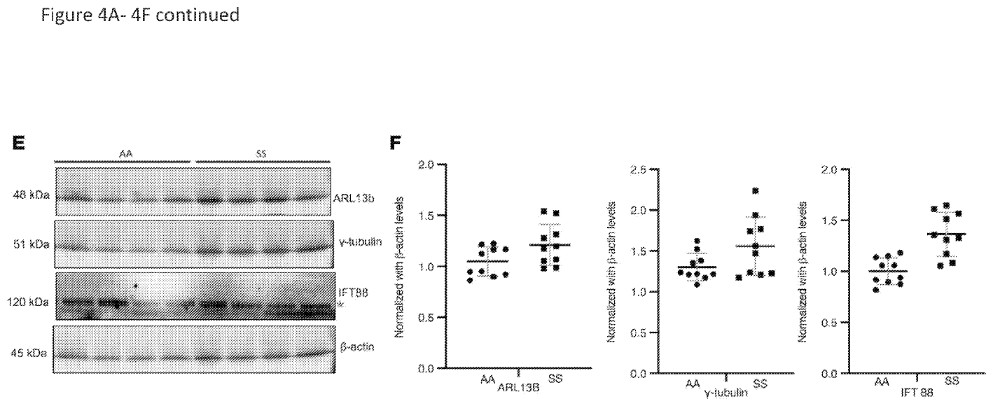

Ciliary length is often correlated with mechanosensory action in blood vessels, with cells experiencing low shear stress having longer cilia and cells in blood vessels with high shear stress having shorter cilia or no cilia. Cilia consist of an axoneme that projects into the lumen and are anchored to the cell through transition fibers that connect to the basal body, a centriole-derived structure. Recent work suggests that cilia can be physically removed from mammalian cells, a process called deciliation, which releases the ciliary membrane and axoneme from the basal body. The Examples demonstrate that the detection or quantification of ciliary proteins in circulation may serve as a biomarker for pathologic conditions of altered flow or changes in blood viscosity. The Examples show that compared with steady-state controls, high shear stress on endothelial cells resulted in about 20% less expression of any cilia-associated proteins after 10-minute perfusion (FIG. 1). The reduction of protein expression in the groups was even pronounced (>50%) at 24 hours postperfusion (FIG. 1). Further, in the cerebral and systemic vasculature, occlusive events in SCD have been postulated to be initiated by sickle RBCs' adhesion to the endothelium. The Examples demonstrate that ciliary proteins may be high in plasma from patients with SCD. Plasma from 10 patients with SCD and 10 healthy individuals using Western blot for the presence of ciliary proteins ARL13b, γ-tubulin, and IFT88 (FIG. 4, E and F) were tested. All 3 ciliary proteins were enriched in the plasma from patients with SCD compared with healthy volunteers (FIG. 4F), demonstrating pathologic condition predisposed to vascular occlusion and/or compromised blood flow, ciliary proteins are present on the surface of RBCs, further accumulate on RBCs upon EC contact, and are enriched in plasma. Cilia proteins may be used as biomarkers for diagnosis of flow-mediated alterations of the vascular endothelium.

In one embodiment, cilia-specific proteins can be quantified in the plasma as biomarkers of endothelial damage or dysfunction. The Examples detected ciliary fragments in effluents in cell culture from ECs and epithelial cells and were able to detect fewer cilia-expressing cells in vivo in ECs. Further, human sickle RBCs displayed enhanced ARL13b ciliary protein on their surface compared with normal RBCs, and once they encountered brain ECs under intermittent flow conditions, the presence of the ciliary protein on these sickle cells was 2.3-fold higher. These results concur with the detection of ciliary proteins at higher levels in SCD plasma compared with healthy plasma. Interestingly, we observed all 3 representative components of the ciliary structure—namely axoneme, transition zone, and basal body—in the plasma of patients with SCD. For SCD, the identification of enhanced cilia protein on RBCs may prognosticate adverse events such as vascular wall weakening, and susceptibility for hemorrhage, both clinically relevant features observed in patients with SCD. Having this information is beneficial for making informed clinical decisions.

### Methods

In one embodiment, the disclosure provides a method of detecting endothelial damage or dysfunction or vascular injury in a subject in need thereof. The method comprises detecting one or more markers of cilium in a biological sample from the subject. As shown in the examples, a higher level of cilium detected in the biological sample compared to control indicates endothelial damage or dysfunction or vascular injury in the subject. The method can then further comprise administering a therapeutic that can treat the endothelial damage or dysfunction or vascular injury, or monitoring the vasculature of the subject (e.g., MRI, etc.).

Endothelial damage or dysfunction is characterized by a loss of barrier function and an infiltration of cellular material into the vascular wall and loss of physiological vascular tone. There is a loss of nitric oxide mediated physiological vasodilation, increased endothelial adhesion and migration of leucocytes and macrophages into the subendothelial vascular wall. Hypoxia, shear forces and oxidative stress trigger events for endothelial dysfunction. Among other things, it leads to a situation in which vasoactive substances, like acetylcholine or serotonin, which normally produce vasorelaxation, cause vasoconstriction. Disorders associated with endothelial dysfunction include; hypertension, atherosclerosis, diabetes, immune system dysfunction, infections, inflammations, cardiovascular disease, stroke, sickle cell anemia, artery-vein malformations, varicose veins, altered tumor vasculature, Thrombotic Thrombocytopenic Purpura (TTP), hemorrhages and preeclampsia.

Vascular injury is any disruption to blood vessel function including those that cause loss of blood, blood clot, bruising, swelling, soreness, pain or swelling.

As used herein, a “subject” may be interchangeable with a “patient” or “individual” and means an animal, which may be a human or non-human animal, in need of treatment. In particular embodiments, the subject is a human subject. A subject in need may be any subject wherein endothelial injury or disfunction may have occurred, including but not limited to subject suspected of having, or diagnosed with hypertension, atherosclerosis, diabetes, immune system dysfunction, infections, inflammations, cardiovascular disease, stroke, sickle cell disease, sickle cell anemia, artery-vein malformations, varicose veins, altered tumor vasculature, hemorrhages and preeclampsia. In one example, the subject has sickle cell disease.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In some embodiments, the biological sample is a biopsy (such as a tumor biopsy). A biological sample may be obtained directly from a subject (e.g., by blood or tissue sampling) or from a third party (e.g., received from an intermediary, such as a healthcare provider or lab technician). In some embodiments, the biological sample is selected from the group consisting of tissues, cells, biopsies, blood, lymph, serum, plasma, urine, saliva, mucus and tears. In certain embodiments, the biological sample comprises a biopsy.

As used herein, the term “marker” or “biomarker” refers to a biological molecule present in a subject at varying concentrations useful in predicting the risk or incidence of a disease or a condition. For example, the biomarker can be a protein present in higher or lower amounts in a subject at risk for endothelial damage or vascular injury. The biomarker can include nucleic acids, ribonucleic acids, or a polypeptide used as an indicator or marker for endothelial damage or vascular injury in the subject. In other embodiments, the marker may comprise markers of cilium. In other embodiment the marker may be a basal body cilium marker. In particular embodiments, the markers may comprise ADP-ribosylation factor-like protein 13B (ARL13b), Intraflagellar transport protein 88 homolog (IFT88), acetylated-α tubulin, Nuclear factor-erythroid factor 2-related factor 2 (NRF2), and one or more basal body marker selected from γ-tubulin or Inversin, and any suitable combination thereof. In some embodiments, the methods comprise detecting two or more cilium markers, alternatively three or more cilium markers.

In some aspects, the one or more markers of cilium is a ciliary marker or a basal body cilium marker. In some embodiments, the detecting step detects at least one ciliary marker and at least one basal body marker. The one or more markers may be selected from the group consisting of ARL13b, γ-tubulin, IFT88, Inversin, nuclear factor-erythroid factor 2-related factor 2 (NRF2), and acetylated-α-tubulin. In one example, the one or more ciliary markers is selected from ADP-ribosylation factor-like protein 13B (ARL13b), Intraflagellar transport protein 88 homolog (IFT88), acetylated-α tubulin, and Nuclear factor-erythroid factor 2-related factor 2 (NRF2), and the one or more basal body marker selected from γ-tubulin or Inversin.

The term “control” for measuring the level of the one or more markers may refer to a sample from a non-diseased patient, a positive control with a known amount of the marker, or may refer to a first sample taken from the same patient from an earlier timepoint that can be used to monitor the change in levels of the marker over time. One skilled in the relevant art is able to pick the proper control to be able to properly measure the level of the markers.

For example, the method may further comprise detecting the one or more markers of cilium in a second biological sample from the same subject, wherein the biological sample is taken at a later time than the first sample. In some aspects, an increase in the level of cilium protein in the second sample as compared to the first sample may indicate endothelial damage or dysfunction or vascular injury in the subject. Therefore, it is contemplated that the cilium markers described herein can be monitored and detected over time, to give an assessment of the risk of or the development of endothelial damage or dysfunction or vascular injury in the subject.

In some embodiments, the detecting one or more markers comprises contacting the sample with one or more antibodies to a marker of cilium, and detecting the presence of the antibody in the sample. Suitable antibodies to markers of cilium can be found in the art, or made by using a laboratory animal to make suitable monoclonal antibodies.

Suitable antibodies are found commercially. Primary antibodies used herein include ARL13b (Proteintech catalog 17711-1-AP), acetylated tubulin (Sigma catalog T6793), IFT88 (Thermo Fisher Scientific catalog PA5-18467), Inversin (Proteintech catalog 10585-I-AP), Dynein (Thermo Fisher Scientific catalog MA1-070), gTubulin (GeneTex catalog GTX113286), Alk1 (Abcam catalog ab51870), KLF4 (Proteintech catalog 11880-1-AP), HO-1 (BD, catalog 566391), NRF2 (BioLegend, catalog 939202), and bactin (Sigma catalog A5441 and Cell Signaling Technology catalog 4970P). Secondary antibodies used are goat anti-mouse PECy7 (BioLegend), donkey anti-rabbit PE (Thermo Fisher Scientific), donkey anti-goat AF657 (Thermo Fisher Scientific), donkey anti-rabbit BV421 (BioLegend), and donkey anti-rabbit AF488 (Thermo Fisher Scientific). However, this disclosure is not limited by these antibodies, and any suitable antibodies that are specific and sensitive and capable of strongly binding to cilium markers can be used in the practice of the disclosure described herein.

Suitably, the primary antibody that is capable of binding the marker of cilium may be directly conjugated to a detectable marker. In other instances, a secondary reagent or secondary antibody capable of binding the first antibody can be used, where the secondary antibody is conjugated to the detectable marker. In some embodiments, a control protein is also used as a measure for quantification of the marker, e.g., the control protein is tested at multiple amounts and a curve is produced correlating the brightness of a signal to the amount of protein to correlate the amount of the cilium marker in the sample. These techniques are well known in the art.

Suitable detectable markers are known in the art and include, for example, fluorescent proteins (green fluorescent proteins (GFP), red fluorescent protein (RFP, dsRed, etc.), yellow fluorescent protein (YFP) EBFP, ECFP, mHoneydew, mBanana, mOrange, tdTomato, mTangerine, mStrawbery, mCherry, mGrape, mRaspberry, mPlum, etc. and include all known in the art, for example, those described in Fluorescent Proteins and Their Applications in Imaging Living Cells and Tissues Dmitriy M. Chudakov, Mikhail V. Matz, Sergey Lukyanov, and Konstantin A. Lukyanov, Physiological Reviews 2010 90:3, 1103-1163, incorporated by reference in its entirety), reporter enzymes, ligand/substrate binding (biotin/streptavidin), and others known in the art.

When using a reporter enzyme, detection is accomplished by measuring the activity of the reporter enzyme via incubation with the appropriate substrate to produce a measurable product. Suitable enzyme labels are horseradish peroxidase (HRP) and alkaline phosphatase (AP). Other suitable enzymes include, but are not limited to, 0-galactosidase, acetylcholinesterase, and catalase. The choice of substrate depends upon the required assay sensitivity and the instrumentation available for signal-detection (spectrophotometer, fluorometer, or luminometer).

In some embodiments, the method of detecting is detecting using an enzyme-linked immunoassay (ELISA). ELISA techniques are readily known in the art. The ELISA may be a direct ELISA, an indirect ELISA, or a sandwich ELISA, among others. In some aspects, the direct or indirect immobilization of markers on a surface can be used. In other embodiments, the primary or secondary antibody bay be immobilized on a suitable surface.

In another embodiment, the detecting is detecting using flow cytometry using beads in which the marker is conjugated, for example, by binding of the cilium marker to an antibody conjugated to the bead, or other suitable methods known in the art.

The methods described herein may have an additional washing step. The washing step may be added to remove any addition of irrelevant protein or other molecules that do not specifically bind to the antibody or reporter marker.

The detection may include measuring the signal generated via the direct or secondary reporter marker in the assay.

In some embodiments, the method further comprises administering a therapeutic to treat the endothelial damage or vascular injury. As used herein, the terms “treating” or “to treat” each mean to alleviate symptoms, eliminate the causation of resultant symptoms either on a temporary or permanent basis and/or to prevent or slow the appearance or to reverse the progression or severity of resultant symptoms of the named disease or disorder.

The endothelial damage or dysfunction or vascular injury that is being detected may be associated with a disease or disorder. The selected from sickle cell anemia, atherosclerosis, stroke, artery-vein malformations, varicose veins, altered tumor vasculature, hemorrhages, preeclampsia, and hypertension. Suitably, the endothelial damage may be associated with preeclampsia, polycystic kidney disease, hypertension, and stroke.

Sickle cell disease us a group of blood disorders, with the most common being sickle cell anemia. Sickle cell disease causes vaso-occlusion which can lead to pain, ischemia and organ damage. Suitable treatments include medications to reduce pain and blood transfusions, among others.

Atherosclerosis or atheriosclerotic vascular disease is a disease in the wall of the artery that leads to lesions and narrowing of the artery. Damage to the endothelium upsets the balance between vasoconstriction and vasodilation and initiates a number of events/processes that promote or exacerbate atherosclerosis; these include increased endothelial permeability, platelet aggregation, leukocyte adhesion, and generation of cytokines. Suitable treatment for atherosclerosis may be dietary changes, therapeutics (e.g., statins and other cholesterol medications, including, for example, a cholesterol absorption inhibitor called ezetimibe (Zetia)), blood thinners (e.g., Warfrin, aspirin, coumadin, etc.), blood pressure medications, surgical procedures (angioplasty and stent placement, endarterectomy, fibrinolytic therapy.

Stoke is a medical condition in which poor blood flow to the brain causes cell death. Endothelial dysfunction occurs after stroke and leads to oxidative stress, inflammation, increased vascular tone, blood-brain barrier (BBB) damage, and further thrombovascular complications in the brain. Suitable treatments are known, including, blood thinners. Stroke is associated with blood components leaking into the brain and can be prognosticated in advance by the cilia biomarker test, allowing for preventative measures (e.g., blood thinner treatment) before adverse effects.

Arteriovenous malformations (AVMs) happen when a group of blood vessels in your body forms incorrectly. In these malformations, arteries and veins are unusually tangled and form direct connections, bypassing normal tissues. Cilia protein levels monitoring will allow for detection of changes in the flow in the lesion, which is now diagnosed by imaging or after the flow is leaked out. Therapy treatment such as inclusion of coils and such and their effect on the malformation could also be determined by cilia biomarker detection kit.

Varicose veins, also known as varicoses, are a medical condition in which superficial veins become enlarged and twisted. Dysfunctional endothelium has a pivotal role perpetuating the inflammatory cascade, with consequent pathological venous changes and chronic venous disease worsening. Endothelial dysfunction may be the central player in the link between varicose veins and deep vein thrombosis. Current treatments are surgery, laser treatment and blood thinning medications. The effects of these treatments can be evaluated and monitored by the methods described herein for detecting the ciliary biomarker profile from blood.

Hemorrhage, is blood escaping from the circulatory system from damaged blood vessels and compromised and permeable endothelium. This will result in close monitoring and additional preventive clinical measures to stop the impending crisis.

Pre-eclampsia is a disorder of pregnancy characterized by the onset of high blood pressure. The increased blood pressure in mother leads to preterm birth of the child. Thus, detecting increased blood pressure changes early with cilia biomarker will alter the course of treatment. Endothelial dysfunction results in hypertension and many of the other symptoms and complications associated with pre-eclampsia. Pre-eclampsia can be treated by early delivery of the baby, hypertensive drugs, among others.

Hypertension, also known as high blood pressure (HBP), is a medical condition in which the blood pressure in the arteries is persistently elevated. Suitable treatments are known in the art, and include, for example, blood pressure medicines capable of lowering blood pressure. Hypertension will lead to more cilia protein in blood, and thus lead to changes in medication regimen.

Polycystic kidney disease is associated in patients who are prone to ballooning of blood vessels in the brain and are monitored for vessel break and bleeding. Blood thinners and anti-hypertensive drugs are used. With a cilia test in hand, blood vessels prone to rupture are likely to increase cilia protein and deposition in the blood, which will alter the course of treatment.

Brain Injury can be monitored using the cilia biomarker methods described herein. The method can determine whether a patient needs an MRI test. This has profound implications for return to work and save MRI costs or prioritize which patients needs immediate attention in ER. Other benefits of cilia biomarker test include minimize “brain radiation” exposure to children and teens. The cilia test could also be used to determine if the treatment is working for brain injury, and how effective the treatment is.

The methods described herein can be used for treating tumor vasculature. Vessels in tumor are tortuous and have abnormal flow. Thus, normalizing flow will help drugs penetrate the tumor. However, whether flow is normalized or not is currently determined by imaging. A cilia biomarker test will help assess the efficacy of flow in the tumor bed post treatment. Also, cilia are longer on tumor cells resistant to therapy. There is no reliable detection platform for resistant cells. Cilia analysis on these cells using the biomarker kit may inform the resistant populations, which can then be targeted for killing.

The biological sample is a sample taken from a subject. For example, suitable biological samples can be serum, blood, urine, cerebrospinal fluid, seminal fluid, saliva, tears, synovial fluid, breast milk, bile, amniotic fluid, aqueous humor, vaginal lubrication, sweat, lymph, and bone marrow. In a preferred embodiment, the biological sample is a blood or plasma sample.

In another embodiment, the disclosure provides a method of detecting an occlusive event associated with sickle cell disease (SCD) in a subject having SCD. The method comprises detecting one or more markers of cilium in a biological sample from the subject. The disclosure also provides methods of monitoring the levels of cilium markers within biological samples taken from a subject over time. For example, the method may further comprise detecting the one or more markers of cilium in a second biological sample from the same subject, wherein the biological sample is taken at a later time than the first sample, and wherein an increase in the level of cilium protein in the second sample as compared to the first sample indicates an occlusive event. Therefore, it is contemplated that the cilium markers described herein can be monitored and detected over time, to give an assessment of the risk or the development of occlusive event.

The detecting one or more markers can comprise contacting the sample with one or more antibodies to a marker of cilium, and detecting the presence of the antibody in the sample. In some aspects, the method further comprises contacting the sample with a secondary antibody with a detectable marker, and detecting the detectable marker in the sample. In some examples, the first or secondary antibody are attached to a solid support. In some aspects, the method includes ELISA or flow cytometry, among others.

In some embodiments, the method further comprises, administering a therapeutic to treat the occlusive event in the subject having SCD.

The term “occlusive event” comprises vascular wall weakening or increased susceptibility to hemorrhage, among other characteristics. Suitable treatments include, but are not limited to, for example, medications to reduce pain (NSAIDS, aspirin, narcotics, etc.) and blood transfusions, among others. Further, scans may be recommended (e.g., MRI, etc.) For example, if cilia levels are high, a scan may be recommended because the aneurysm (dilated blood vessel) may be showing signs of breakage, which would not be good for the patient health. Currently, they routinely scan the patient for such dilated vessels and hope that over time it does not get worse. These scans are done on routine scheduled visits to the clinic. Thus, saving unnecessary scans by ordering our tests will be beneficial. Also, scanning for when need arises instead of routinely would reduce the number of scans a subject is exposed and provide anon-invasive means for monitoring a subject. Additionally, changing the course of treatment based on the impending crisis is benefit clinically as well by identifying increased cilia.

In another embodiment, the disclosure provides a method of detecting cilia on red blood cells in a sample from a subject, the method comprising obtaining a blood sample from a subject; isolating the red blood cells (RBCs) from the sample, and detecting one or more markers of cilium on the surface of the red blood cells. The detection of cilium on the RBC is indicative of damaged blood vessels. The method may further comprise obtaining a second blood sample from a subject; isolating a second set of red blood cells from the sample, detecting one or more markers of cilium on the surface of the RBCs, and comparing the level of cilium on the RBCs from the first sample to the second sample, where the second sample is taken at a later time than the first sample, and wherein the increase in cilium associate with the RBCs indicates damage to one or more blood vessels within the subject. The first and second sample may be taken weeks, months or years apart for monitoring. Suitably the method may comprise taking a third, fourth, fifth, sixth, etc. sample and comparing to the earlier samples to monitor changes in cilia concentration in the samples and alter to any changes within the subject. For example, a sample may be taken monthly to monitor a condition described herein. In some examples, the samples may be taken weekly (e.g., preclamsia) to monitor the pregnant subject.

### Kits:

In another embodiment, the present invention provides kits for carrying out the methods described herein, including kits for measuring the cilium marker in a biological sample. Suitably, the kit can contain at least one antibody that binds to at least one cilium marker, and instructions for use. The kit may further comprises at least one secondary antibody with a detection label. The kit contemplated herein can contain one or more antibodies that bind to a cilium marker, the cilium marker selected from the group consisting of ARL13b, γ-tubulin, IFT88, Inversin, nuclear factor-erythroid factor 2-related factor 2 (NRF2), and acetylated-α-tubulin.

In another embodiment, an ELISA kit to detect cilium is provided. The ELISA kit comprises a solid support, at least one primary antibody that binds specifically to cilium, at least one secondary antibody specific to the primary antibody and having a detectable marker, and instructions for use. The detectable marker can be an enzyme reporter. The detectable marker can be a fluorescent marker or a colorimetric marker. In one aspect the ELISA kit can screen for the detection of basal body of cilium based on proteins expressed on cells of origin of cilium using the methods described herein. The kit would comprise one or more antibodies specific to the basal body of cilium, described herein. In another aspect, a combination ELISA kit to screen both cilium and basal body of cilium markers can be provided. The kit can include at least one ciliary marker and at least one basal body marker antibody, and methods of detecting both markers separately (e.g., different reporter molecules for each marker). In another aspect, the kit may be an immunofluorescent detection kit to detect cilium and basal body for basic research or clinical samples, the kit comprising one or more antibodies that bind to cilium markers. The primary antibodies may be directly conjugated to a fluorescent molecule, or a fluorescently conjugated secondary antibody may be used. Suitable antibodies are known and described herein.

The kits and methods described herein provide a benefit over prior methods of detection. Because ECs experience altered flow or shear stress in various pathophysiologic conditions, such as preeclampsia, polycystic kidney disease, hypertension, and stroke to name a few, circulating ECs have been considered as possible biomarkers of vascular insult or endothelial dysfunction. However, due to their low numbers in circulation, and difficulty in detection, their application has been limited. On the other hand, as shown here, cilia from damaged ECs can be detected readily in circulation and biological samples. This method is a better alternative to detecting circulating ECs given that blood components are the only point of contact for EC cilia expressed on the luminal side. Also, given that cilia are expressed in most EC beds and that flow influences cilia integrity, any condition where flow is compromised constitutes an opportunity for the application of cilia biomarkers, broadening the value of cilia biomarkers for vascular injury. At minimum, the ability to detect ciliary proteins in blood or other body fluids and in various conditions influenced by flow provides an additional tool in the clinical toolbox to inform the physician of a possible pathology. In summary, our work warrants extended investigation to understand whether and how cilia-specific proteins in circulation can be developed into prognosticative markers of disease where the flow-related homeostasis of the endothelium is compromised.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: Cilia Proteins are Biomarkers of Altered Flow in the Vasculature

Cilia are microtubule-based organelles that are present in most eukaryotic cells (1) and are distinguished based on the arrangement of 9 outer microtubule doublets enclosing a central doublet (9+2) or not (9+0). 9+0 cilia are often referred to as primary nonmotile cilia and 9+2 cilia as motile cilia (2). However, these definitions need revisiting given the recent evidence suggesting that mixed cilia (motile and nonmotile) can be found in eukaryotic cells (3). Cilia function as mechanosensors for cells. In endothelial cells (ECs) that line the vasculature, the 9+0 cilia are often found on the apical (luminal) surface and are thought to sense blood flow and transduce these mechanical signals into chemical signals inside the cells that control shear-responsive behavior (4, 5). Primary endothelial cilia's role as mechanosensors has been reported in mouse aortic ECs in vitro, isolated mouse arteries, blood vessels from human placenta ex vivo, and in vivo mouse models (6-10). Shear stress from physiologic blood flow elicits cilia bending (11). Shear stress, the tangential force of blood flow on the surface of the endothelium, normally varies throughout the macro- and microvasculature and can drastically vary in different physiologic and pathologic conditions. Ciliary length is often correlated with mechanosensory action in blood vessels, with cells experiencing low shear stress having longer cilia and cells in blood vessels with high shear stress having shorter cilia or no cilia (12, 13). In both zebrafish and mammals, primary cilia are considered enriched in regions of low shear stress (11, 14). At low shear stress, in the mammalian retinal vasculature, primary cilia are suggested to act in concert with bone morphogenetic protein 9 to minimize vessel regression before onset of high shear stress-mediated vascular remodeling (15). Thus, collectively, EC cilia are widely considered as low shear stress sensors.

In our own work, we have corroborated some of these initial findings and identified EC cilia in regions of low shear stress, such as bifurcation junctions in the juvenile zebrafish vasculature (16). Thus, the question arises as to what happens to EC cilia under increased shear stress conditions. Seminal work from Iomini et al. shows disassembly of primary cilia from human umbilical vein ECs (HUVECs) experiencing laminar shear stress of 15 dyne/cm² for 1 hour (13). Cilia consist of an axoneme that projects into the lumen and are anchored to the cell through transition fibers that connect to the basal body, a centriole-derived structure (1). Recent work suggests that cilia can be physically removed from mammalian cells, a process called deciliation, which releases the ciliary membrane and axoneme from the basal body (17). Intact shed cilia were recovered in culture media and contained both membrane and axoneme fragments. Further, this deciliation process was reported to be rapid and was suggested as the predominant mode of cilia loss in mammalian cells. In a separate study, whole cilia or partial ciliary fragments were observed in urine from mice subjected to chemically induced acute kidney injury (18) presumably from kidney epithelial cells, thus attributing this phenomenon to multiple cell types and tissues. The consequence of high shear stress-induced deciliation in embryonic ECs is directly associated with vascular instability and hemorrhage in embryonic zebrafish (16, 19-21) and vascular barrier integrity (22, 23). In mice (24, 25), ciliary mutants show extensive hemorrhages. These studies collectively suggest the hypothesis that cilia in ECs are disassembled (deciliation) upon disruption of vascular homeostasis, thus causing release of the ciliary fragments into circulation. Detection or quantification of ciliary proteins in circulation may serve as a biomarker for pathologic conditions of altered flow or changes in blood viscosity. In this study, we tested this hypothesis using a combination of cellular, vertebrate, and human model systems.

### Results

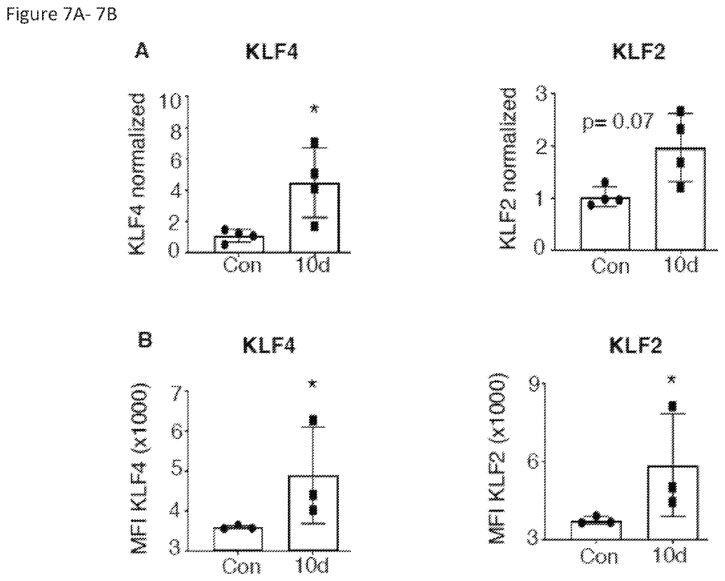

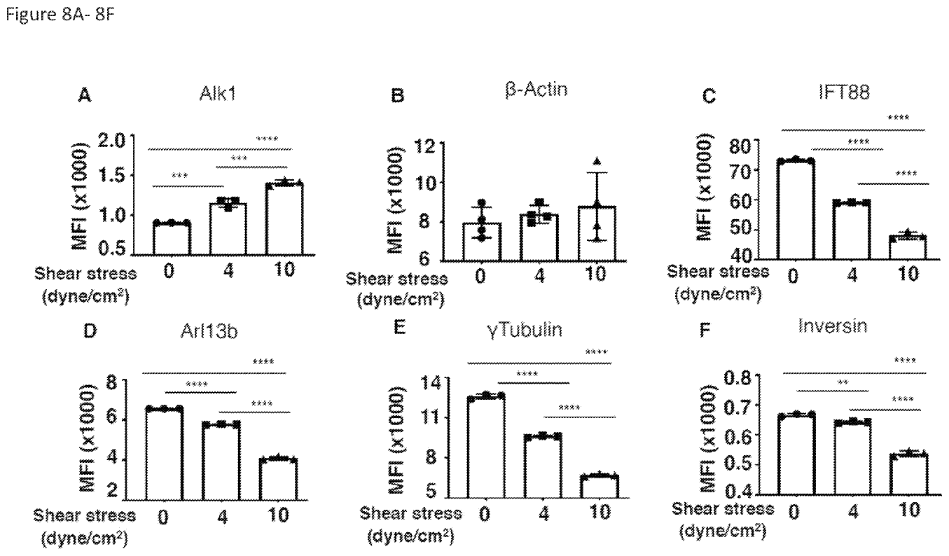

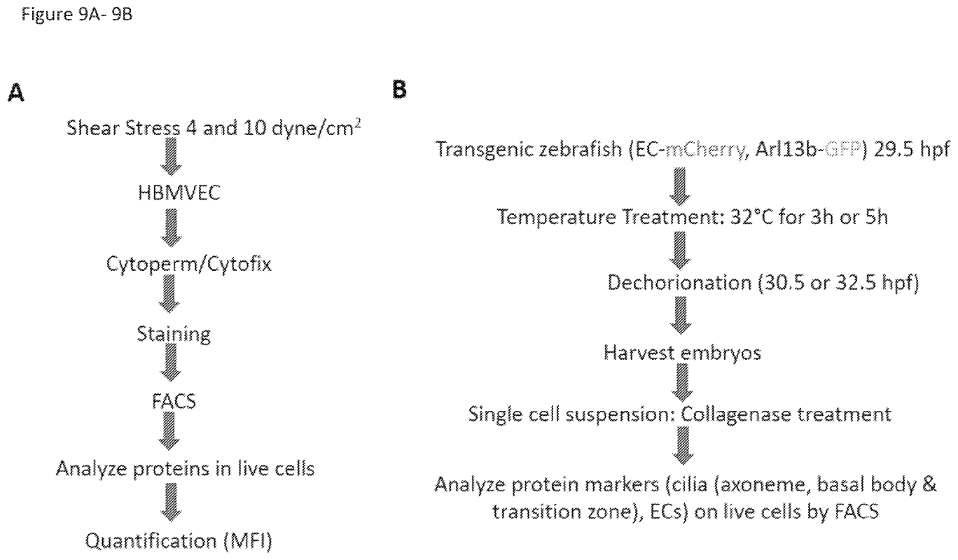

Increased shear stress in ECs facilitates deciliation in vitro. To test the effect of shear stress on EC cilia in vitro, we chose cells from a microvascular brain vessel bed, human primary brain microvascular ECs (HBMVECs), and a macrovessel venous bed, HUVECs. We applied laminar flow shear stress of 0, 2, 4, and 10 dyne/cm² on HBMVECs, which was reported previously (26), and used 2 different methods for subjecting the cells to shear stress. In a microfluidic device-based unidirectional shear stress method (Ibidi system), we used graded strengths of shear stress starting at 2 dyne/cm² and increased to 4 and 10 dyne/cm² for 10 minutes and 24 hours (FIG. 1). With a shaker method, which is based on circulatory motion-associated shear stress, we applied 4 and 10 dyne/cm² for 4 minutes (FIG. 8). After shear stress, cells were collected for FACS analysis of specific markers: KLF4, KLF2, ALKI (flow), ARL13b (cilia axoneme), γ-tubulin (cilia basal body), IFT88 (cilia transition zone protein), Inversin (cilia basal body), NRF2 (cilia-related gene), and bACTIN (housekeeping gene) (FIG. 9A). With both methods, we observed the control flow marker expression increase in brain ECs. With the Ibidi method, KLF4 (FIG. 7A) and KLF2 (FIG. 7B) expression were significantly increased in 10 dyne/cm² condition compared with the “no flow” control at 24 hours. With the shaker method, a linear increase in ALKI-expressing cells at 4 and 10 dyne/cm² (P<0.0001) (FIG. 8A) was observed.

With both methods, interestingly, all ciliary markers tested showed reduction in protein levels at 10 dyne/cm² and 4 dyne/cm² (FIGS. 1 and 8, C-F), with no change in housekeeping bACTIN protein expression (FIG. 8B). With Ibidi method, at a shorter period of 10 minutes, compared with 24 hours, lower protein expression levels were generally observed (FIG. 1), which was distinct at 2 dyne/cm², a physiologically steady-state situation. At 10 minutes as well as 24 hours, the 4 and 10 dyne/cm² groups showed significantly reduced cilia-associated protein expression compared with the steady-state control group. Compared with steady-state controls, experimental groups (4 and 10 dyne/cm²) had about 20% less expression of any cilia-associated proteins after 10-minute perfusion (FIG. 1). The reduction of protein expression in the groups was even pronounced (>50%) at 24 hours postperfusion (FIG. 1).

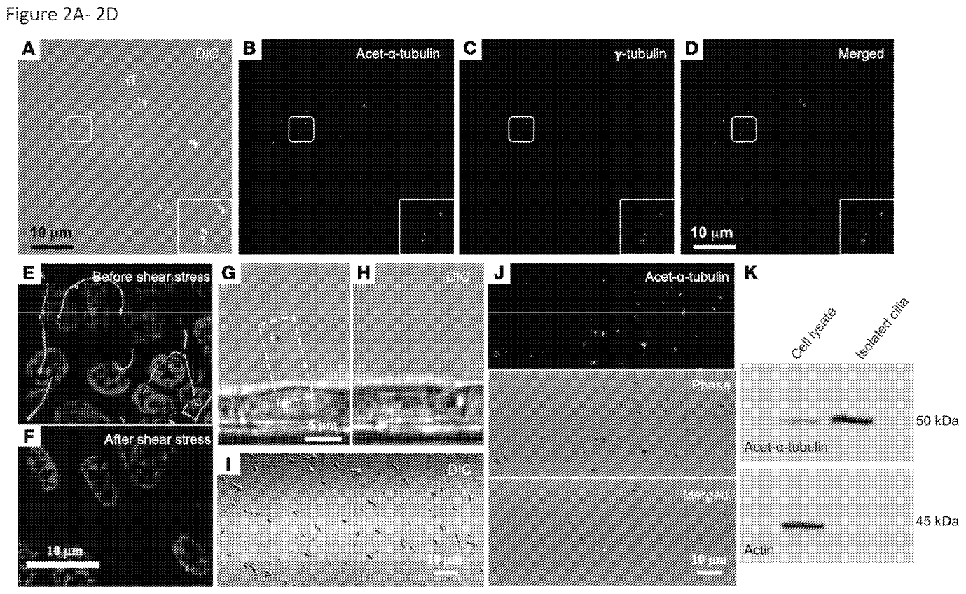

For HUVECs plated on a monolayer, we used the shaker method to apply high shear stress at 20 dyne/cm² as reported previously in the literature (27, 28). Effluents were collected, drop concentrated, and analyzed by differential interference contrast (DIC) microscopy. Staining was performed on the effluents for ciliary marker acetylated-α-tubulin and basal body (γ-tubulin). As shown (FIG. 2, A-D), the effluents showed positivity for both acetylated-α-tubulin (FIG. 2B) and γ-tubulin (FIG. 2C) proteins. We also applied 10 dyne/cm² shear stress to a porcine kidney epithelial cell line, LLC-PK1, using the shaker method and stained for ciliary marker acetylated-α-tubulin (FIG. 2, E and F), which clearly showed loss of cilia marker staining after shear stress. A single cilium observed by phase contrast microscopy (FIG. 2G, white box) was missing after shear stress (FIG. 2H).

Further, effluents (FIG. 2I) collected from these experiments were positive for acetylated-α-tubulin (FIG. 2J), and Western blots for acetylated-α-tubulin clearly showed expression in isolated cilia with no actin protein detected in the same isolate (FIG. 2K). Thus, collectively, these data argue that shear stress of greater than 2 dyne/cm² can influence cilia removal, loss of cilia proteins on cell surface, and presence of cilia proteins and fragments in effluents in macro- and microvessel beds as well as kidney epithelial cells.

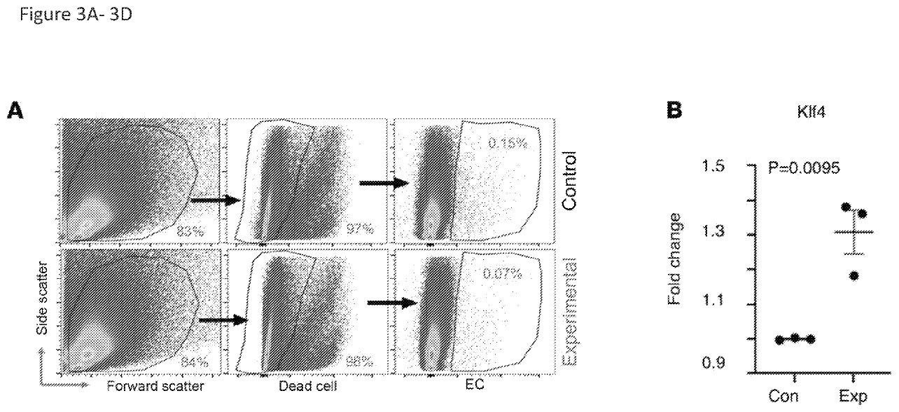

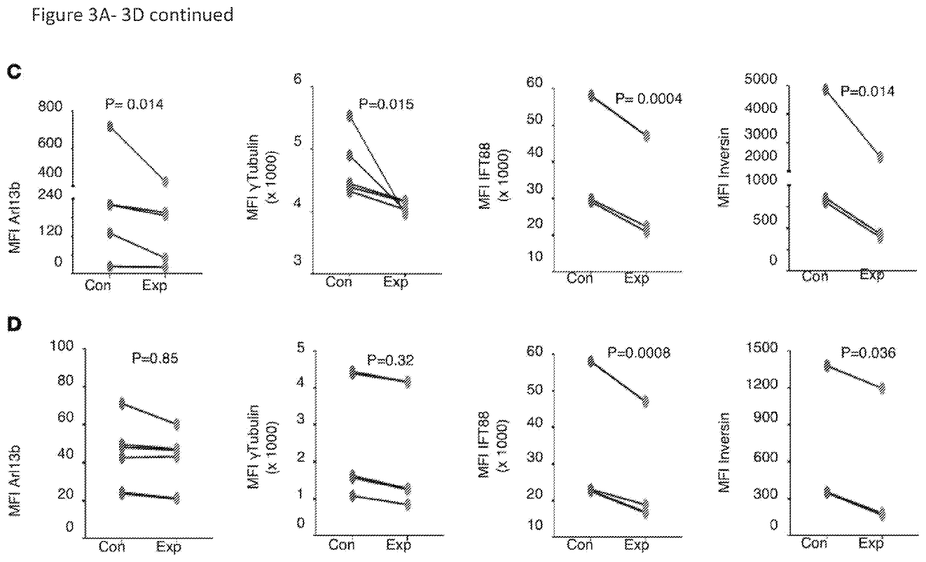

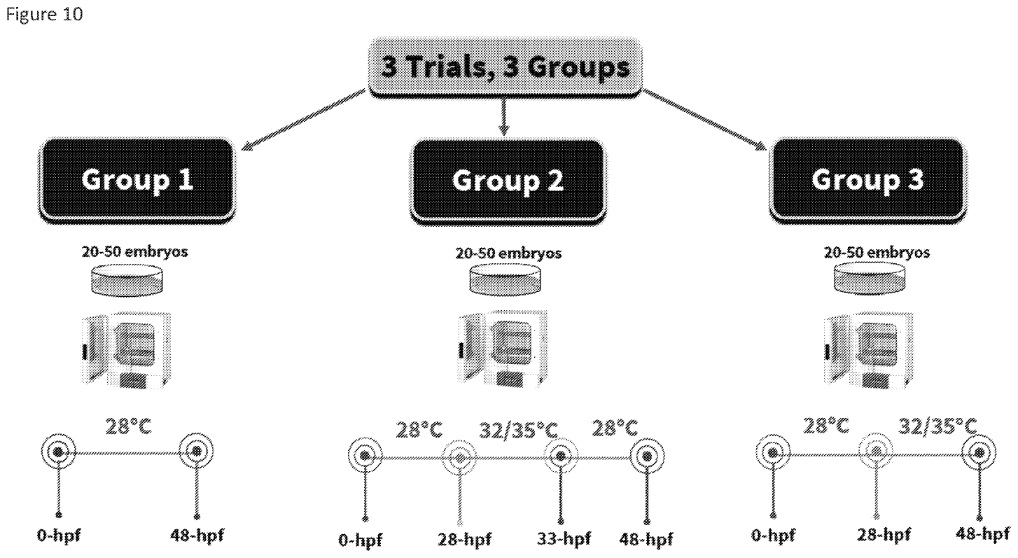

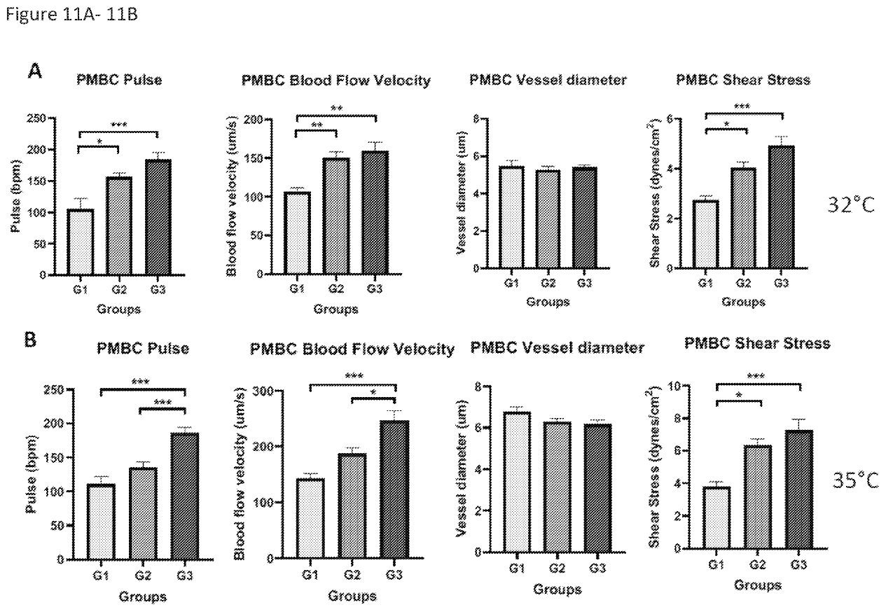

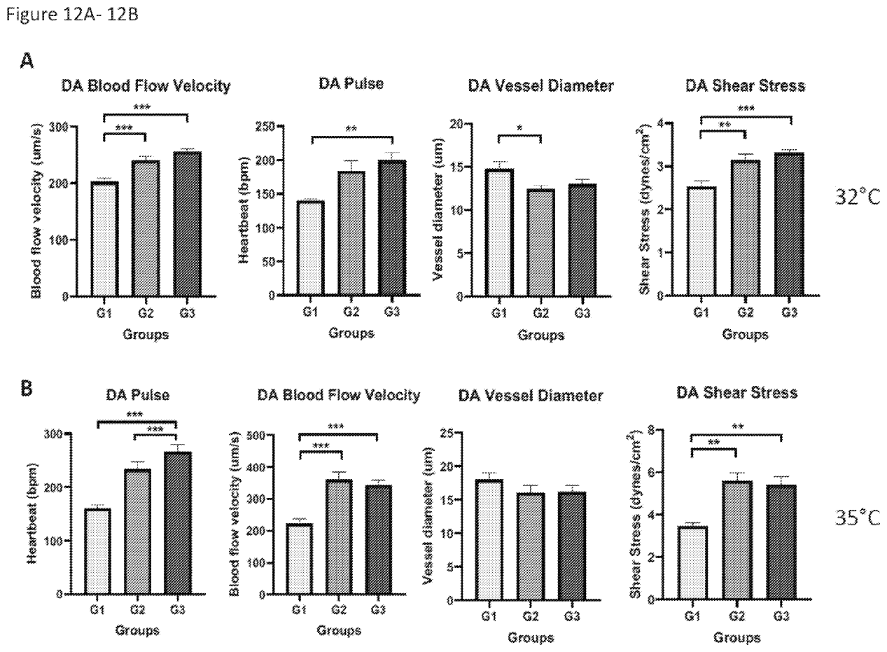

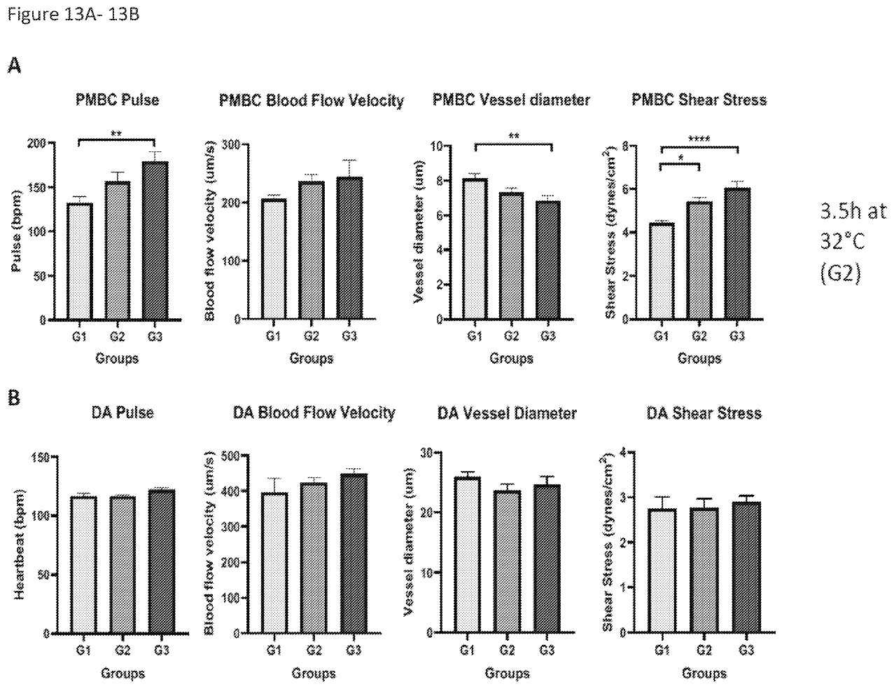

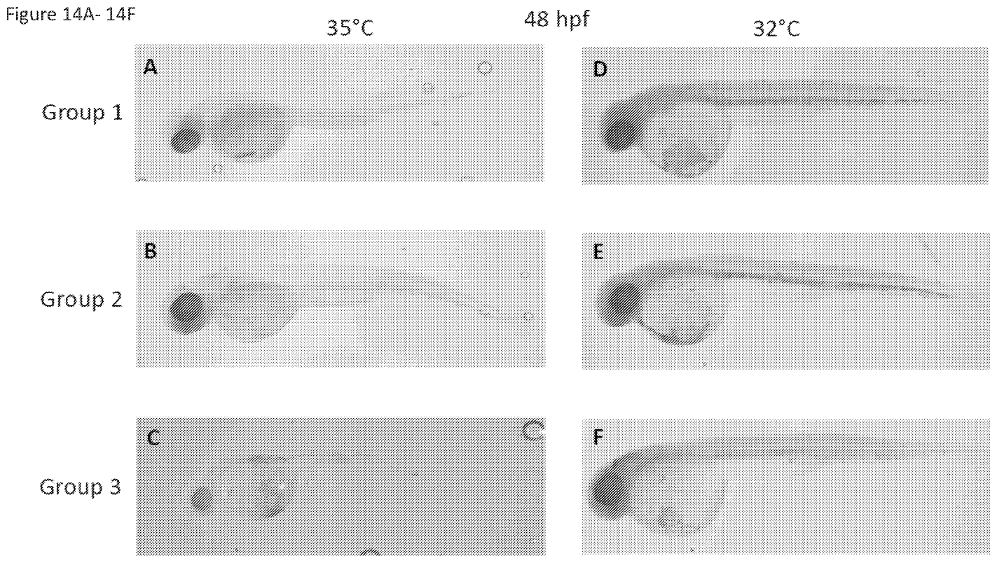

Increased shear stress in vivo results in ECs with lower amounts of cilia protein expression. To test the effect of shear stress on EC cilia in vivo, we took advantage of the versatility of the vertebrate zebrafish model system (FIG. 9B). Previously, others (29, 30) have reported that increasing the incubation temperature of zebrafish embryos from 28° C. to 34.5° C. increases their heartbeat and blood flow rate. Upon increasing temperature, we calculated that the shear stress increased from 2.3 to 3.1 dyne/cm² in the primordial midbrain venous channel (PMBC) (16) and from 2.0 to 2.97 dyne/cm² in the dorsal aorta (DA). However, these measurements were made at 33 hours postfertilization (hpf), after 5 hours of incubation from 28 to 33 hpf at the higher temperature. We performed a systematic analysis of the effect of temperature (32° C. and 35° C.) with shorter (3.5 and 5 hours) and longer (24 hours) incubation periods starting at 28 hpf (blood flow commenced in brain), then measured blood flow-associated parameters in the PMBCs and DAs at 48 hpf (FIGS. 10-13). Three groups of embryos (n=20-50 per group) (FIG. 10) were analyzed in both PMBC (FIG. 11 and FIG. 13A) and DA (FIG. 12 and FIG. 13B) vessels for blood flow (pulse, blood flow velocity, vessel diameter, shear stress) parameters. The samples included group 1 (G1): 0-48 hpf (28° C.); group 2 (G2): 0-28 hpf (28° C.) followed by 3.5 or 5 hours of incubation at 32° C. or 35° C. and returning to 28° C. until 48 hpf, and group 3 (G3): 0-28 hpf (28° C.) followed by incubation at 32° C. or 35° C. until 48 hpf (FIG. 10). We made the following overall observations: (a) the 35° C. over 32° C. temperature showed more robust changes in blood flow parameters (pulse, velocity, vessel diameter, and shear stress) but also induced scoliosis (curvature of spine) in embryos (FIG. 14); (b) irrespective of the vessel caliber or location, for the most part, G3 embryos showed higher blood flow parameters; (c) shear stress values were generally higher for PMBC (3-5 dyne/cm² [32° C.] or 4-7 dyne/cm² [35° C.]) versus DA vessels (2.5-3 dyne/cm² [32° C.] or 3.5-5.2 dyne/cm² [35° C.]); and (d) exposure for 3.5 hours at 32° C. also showed robust changes in the assessed parameters of the PMBC except for blood flow velocity (FIG. 13A), with no significant changes observed in the DA (FIG. 13B). It is noteworthy that increasing shear stress by this method only showed hemorrhages in brain vessels and not the trunk, which is thought to be associated with loss of cilia in ECs (16). Thus, we chose the shorter time point of about 3 hours' incubation (29.5-32.5 hpf) and lower temperature (32° C.) for increasing shear stress in PMBCs of transgenic (Tg) (flk: mCherry; bactin: Arl13b-GFP) zebrafish embryos, where ECs were labeled red and cilia were labeled green. Single-cell suspension and subsequent FACS analysis on live and labeled ECs (FIG. 9B) were performed as described in the Methods section. Markers assessed included Klf4 (flow), Arl13b (cilia axoneme), γ-tubulin (cilia basal body), Inversin (cilia basal body), and Ift88 (cilia transition zone). In the shear stress induction group (experimental), we observed the expected increase in Klf4-expressing MFI in ECs (FIG. 3, A and B). All cilia markers assessed showed a decrease in experimental samples compared with control samples (FIG. 3C) when assessed in the mCherry⁺ (EC) population. In the mCherry (non-EC) population (FIG. 3D), the reduction of the respective cilia-associated protein expressions was less pronounced compared with what was observed in the EC population. These results suggest that upon increased shear stress in brain vessels in vertebrate embryos, ECs express fewer ciliary proteins on their cell surface.

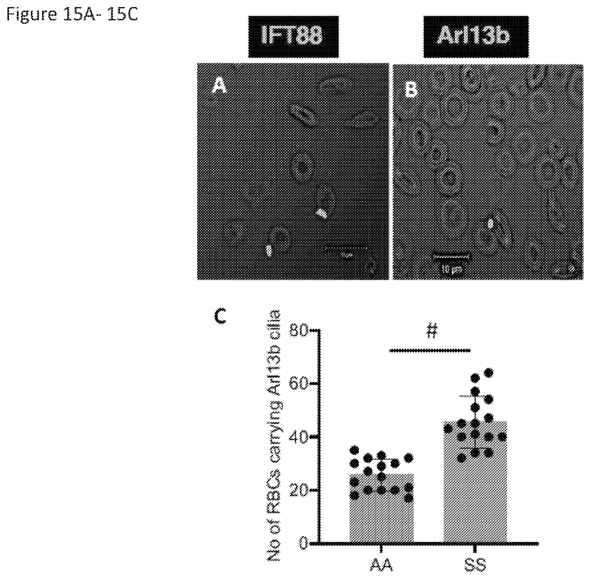

Circulating RBCs adhere to brain ECs and accumulate cilia proteins postadhesion. To investigate ciliary proteins in circulation in a pathophysiologic model with translational and clinical significance, we selected sickle cell disease (SCD). SCD is caused by a single mutation in the β-globin gene that changes the sixth amino acid in the β-globin protein of hemoglobin from glutamic acid to valine (31), which makes RBCs highly susceptible to sickling due to the production of sickle hemoglobin (Hb S) and thus impairs Hb capacity to deliver oxygen to tissues. In the cerebral and systemic vasculature, occlusive events in SCD have been postulated to be initiated by sickle RBCs' adhesion to the endothelium (32). Thus, we hypothesized that sickle RBCs adhere to brain ECs, triggering cilia shedding, and that ciliary proteins would be high in plasma from patients with SCD. We first investigated whether RBCs isolated from patients homozygous for Hb S (SS) would adhere preferentially to a monolayer of brain ECs, compared with healthy volunteers with normal Hb A (AA). A graduated height flow chamber adhesion assay to quantify the adhesion of RBCs to ECs was performed as described previously (33, 34). Indeed, 58%±9% of SS RBCs compared with 7%±1.8% of AA RBCs adhered to brain ECs subjected to shear stress of 1 dyne/cm² (FIG. 4A). Next, we performed flow cytometric analysis on circulating RBCs isolated from SS patients for the presence of cilia protein ARL13b, prior to and following exposure to brain ECs in the flow chamber. Compared with AA RBCs, which showed 1%±0.47% of bound ARL13b, SS RBCs showed a remarkable 23-fold increase to 23%±2.5% of bound ARL13b prior to exposing brain ECs to the AA and SS RBCs, respectively (FIG. 4B). Upon flow exposure, ARL13b presence on AA RBCs was not affected, but it did increase further by 2.3-fold on SS RBCs to 54%±8.8% (FIG. 4C), suggesting that SS RBCs may have collected additional cilia from brain ECs. We performed a blood smear to directly visualize accumulation of ARL13b on SS RBCs (FIG. 4D). A similar blood smear was performed from blood isolated from sickle SS mice and immunostained for ARL13b and IFT88 proteins (FIG. 15). ARL13b- and IFT88-positive cilia-expressing mouse RBCs were found in smears from SS mice (FIG. 15, A and B). Quantification (FIG. 15) showed a 2-fold enrichment of ARL13b-positive RBCs in SS versus AA mouse blood. Finally, we investigated plasma from 10 patients with SCD and 10 healthy individuals using Western blot for the presence of ciliary proteins ARL13b, γ-tubulin, and IFT88 (FIG. 4, E and F). All 3 ciliary proteins were enriched in the plasma from patients with SCD compared with healthy volunteers (FIG. 4F). Taken together, these data sets suggest that in a pathologic condition predisposed to vascular occlusion and/or compromised blood flow, ciliary proteins are present on the surface of RBCs, further accumulate on RBCs upon EC contact, and are enriched in plasma. Thus, collectively, our work suggests cilia proteins as potential biomarkers for diagnosis of flow-mediated alterations of the vascular endothelium.

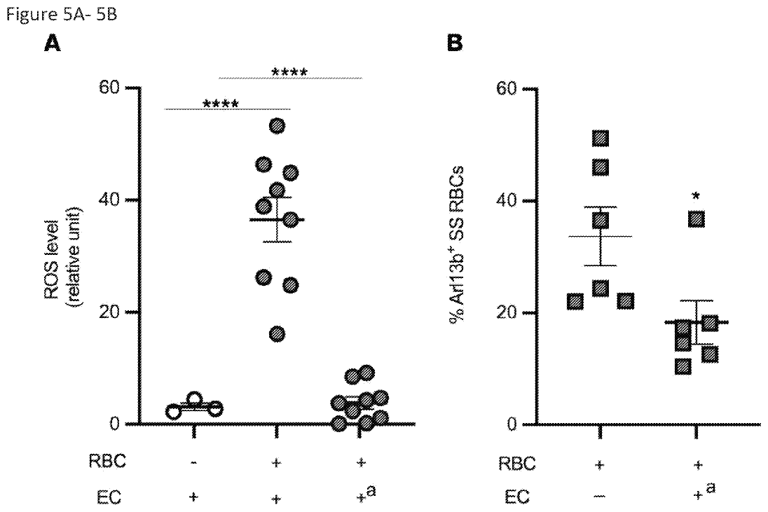

EC cilia stability is dependent on ROS generation in brain ECs. To investigate the underlying mechanism associated with loss of cilia in brain ECs due to shear stress or RBC interaction, we focused on excessive reactive oxygen species (ROS) and oxidative stress. Previous work has suggested that interactions of sickle RBCs with HUVECs induce endothelial oxidative stress (35). Thus, we investigated whether adhesion of SS RBCs from patients with SCD patients to brain ECs triggers ROS generation in ECs. Indeed, SS RBC interactions with brain ECs increased EC ROS levels compared with basal levels of EC ROS (P<0.05; FIG. 5A). Pretreatment of brain ECs with the NADPH oxidase (NOX) inhibitor, apocynin, reduced ROS generation in ECs to baseline levels (P<0.0001; FIG. 5A), suggesting that SS RBC-induced increased ROS production in brain ECs is dependent on activation of NOX enzymes. To investigate the effect of increased ROS levels in ECs on SS RBCs-cilia, in subsequent experiments, we analyzed by flow cytometry SS RBCs for the presence of the ciliary protein ARL13b prior to and following exposure in the flow chamber to brain ECs pretreated with apocynin. Before exposure to apocynin-treated brain ECs, SS RBCs showed approximately 35% ARL13b cilia expression, but after exposure to apocynin-treated brain ECs, SS RBCs showed a decrease of approximately 20% in ARL13b expression (P<0.05, FIG. 5B). We interpret these data to mean that inhibition of NOX enzymes in brain ECs with apocynin prevented SS RBCs from collecting additional ARL13b protein from brain ECs.

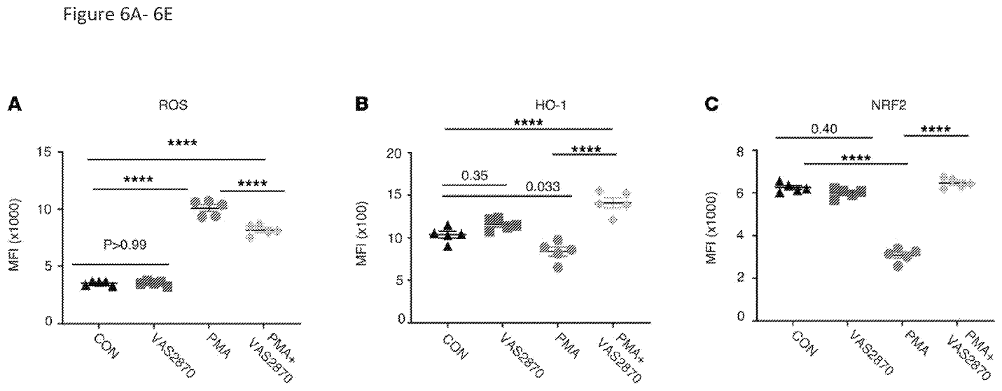

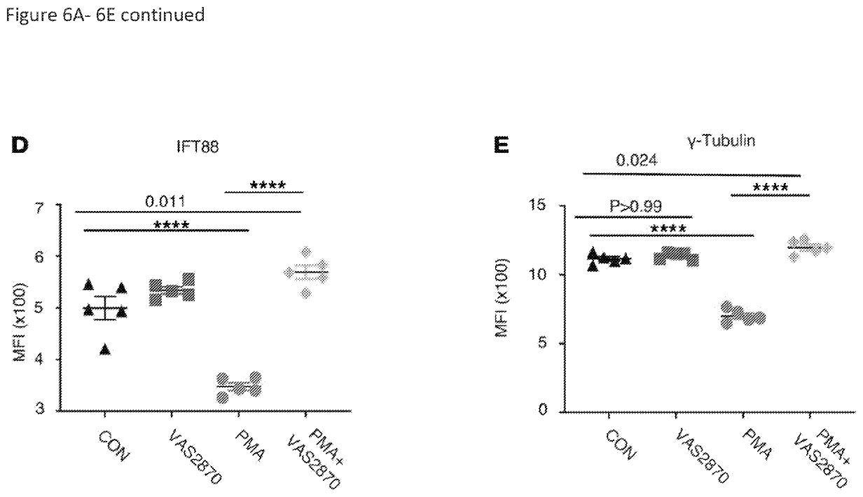

To assess if attenuating oxidative stress in brain ECs will rescue cilia protein levels, we treated human brain ECs with PMA, a known oxidative stress inducer, in ECs in the presence or absence of the NOX small-molecule inhibitor VAS2870. We then quantified the expression of total ROS, heme oxygenase 1 (HO-1, oxidative stress counteractor), and cilia-associated proteins NRF2, IFT88, and γ-tubulin (FIG. 6) levels using flow cytometry method. As expected, PMA induced ROS in brain ECs (FIG. 6A), which was partially attenuated by VAS2870. HO-1 levels (FIG. 6B) were lower in PMA-treated brain ECs, and upon NOX inhibition, levels were restored to control levels. Interestingly, all 3 cilia-associated proteins (FIG. 6, C-E) were reduced upon PMA treatment, and levels were restored to baseline upon NOX inhibition in brain ECs. Taking the sickle RBC ROS data and brain EC-based ROS data together, oxidative stress appears to be one of the key pathways in ECs that is responsible for cilia stability.

### DISCUSSION

Our present study reveals that cilia-specific proteins can be quantified in the plasma and should be explored as biomarkers of endothelial damage or dysfunction. In the ECs lining the vascular wall, cilia have been postulated as a low-flow sensor (11). In support of this hypothesis, cilia are indeed enriched in regions of vessel wall where flow is minimal, such as curvature in the vessel (14, 16). Under steady state, the vasculature experiences a constant flow. In the venous system, an average shear force of 1-4 dyne/cm² is reported while capillaries experience shear from 10-20 dyne/cm² (26). In arteries, shear force varies from 4 dyne/cm² in the common carotid artery to 13 dyne/cm² in the brachial artery (36). We previously demonstrated in zebrafish embryos, that upon incubation of embryos at 34.5° C., 2-4 dyne/cm² shear stress is induced in brain microvessels that results in brain hemorrhage and loss of ciliary structures in primordial midbrain venous channels (16). Others have also reported that loss of ciliary proteins in zebrafish causes brain vessel instability resulting in hemorrhages (19). Further, primary cilia are disassembled when HUVECs (macrovessels) are subjected to 15 dyne/cm² of laminar shear stress (13). Thus, collectively, 4-20 dyne/cm² shear force as observed in vivo is sufficient to make ECs lose ciliary proteins in vitro and was indeed observed in this study. Here, we were able to detect ciliary fragments in effluents in cell culture from ECs and epithelial cells and were able to detect fewer cilia-expressing cells in vivo in ECs subjected to high shear stress.

The question of how cilia on the EC surface may be influenced by altered flow to facilitate detachment remained an open question. In addition to increased or disturbed physical flow, we hypothesized that blood constituents, in particular, RBCs, may also contribute to the deciliation process in ECs (17) in pathologic conditions (FIG. 7). We chose SCD, where the RBC morphology is altered to have a sickled shape and RBCs are known to cause oxidative stress-mediated damage to EC membranes (35, 37, 38). Further, in SCD, blood flow is altered because of enhanced adhesion of sickle RBCs to themselves and the endothelium, promoting vascular occlusion (39), which can subsequently facilitate damage to the endothelium (35). Thus, this pathophysiologic model offers the ideal opportunity to identify cilia proteins emerging from the damaged endothelium that may be the result of sickle RBC adhesion to the endothelium. Remarkably, we found that human sickle RBCs displayed enhanced ARL13b ciliary protein on their surface compared with normal RBCs, and once they encountered brain ECs under intermittent flow conditions, the presence of the ciliary protein on these sickle cells was 2.3-fold higher. These results concur with the detection of ciliary proteins at higher levels in SCD plasma compared with healthy plasma. Interestingly, we observed all 3 representative components of the ciliary structure—namely axoneme, transition zone, and basal body—in the plasma of patients with SCD, suggesting that cilia disassembly is not partial. These results also concur with the recent observations in mammalian cells, where cilia shed from cells expressing mCherry-atubulin (axoneme marker) contained atubulin in the ciliary fragments, suggesting that axoneme is shed together with the ciliary membrane (17).

To investigate the underlying mechanism associated with cilia stability in static and flow-induced sickle RBCs/brain ECs interaction, we focused on oxidative stress and ROS generation (FIG. 7). Human sickle RBCs adhere to brain ECs similar to HUVECs (35) and induce increased ROS generation. Sickle RBCs exposed to brain ECs treated with apocynin, a NOX inhibitor, showed decreased cilia protein expression. These data argue that cilia on sickle RBCs are influenced upon interaction with ROS-quenched brain ECs (FIG. 7). Two interrelated possibilities emerge to explain this result: either ECs retain more cilia when oxidative stress is minimized and thereby RBCs capture fewer cilia from ECs, or RBCs lose cilia when they interact with ROS-inhibited ECs and thus have fewer cilia. It is difficult to differentiate between the two. Under static conditions, brain ECs were responsive to stress inducers such as PMA, and showed enhanced total ROS, which upon treatment with a different NOX inhibitor, VAS2870, quenched ROS production following the PMA-induced oxidative stress. Interestingly, the cilia protein levels that were decreased in PMA-treated brain ECs returned to baseline in NOX inhibitor-treated brain ECs. The static condition result argues against the possibility that ECs' cilia are not available for interaction upon ROS inhibition and supports the second hypothesis that RBCs lose cilia upon interaction with ROS-inhibited ECs. These results collectively argue that cilia stability in both ECs and RBCs is susceptible to increased ROS levels in brain ECs. This interpretation is also in line with previous studies in epithelial cells where they show that, after ischemic injury in murine kidneys, reduction of oxidative stress accelerates the recovery of primary cilia length (40).

For SCD, the identification of enhanced cilia protein on RBCs may prognosticate adverse events such as vascular wall weakening, and susceptibility for hemorrhage, both clinically relevant features observed in patients with SCD. Having this information is beneficial for making informed clinical decisions. Because ECs experience altered flow or shear stress in various pathophysiologic conditions, such as preeclampsia, polycystic kidney disease, hypertension, and stroke to name a few, circulating ECs have been considered as possible biomarkers of vascular insult or endothelial dysfunction (41, 42). However, due to their low numbers in circulation, and difficulty in detection, their application has been limited. On the other hand, as shown here, cilia from damaged ECs can be detected readily in circulation. This method is perhaps a better alternative to detecting circulating ECs given that blood components are the only point of contact for EC cilia expressed on the luminal side. Also, given that cilia are expressed in most EC beds and that flow influences cilia integrity, any condition where flow is compromised constitutes an opportunity for the application of cilia biomarkers, broadening the value of cilia biomarkers for vascular injury. At minimum, the ability to detect ciliary proteins in blood or other body fluids and in various conditions influenced by flow provides an additional tool in the clinical toolbox to inform the physician of a possible pathology. In summary, our work warrants extended investigation to understand whether and how cilia-specific proteins in circulation can be developed into prognosticative markers of disease where the flow-related homeostasis of the endothelium is compromised.

### Methods

### Antibodies

Primary antibodies used in this study include ARL13b (Proteintech catalog 17711-I-AP), acetylated tubulin (Sigma catalog T6793), IFT88 (Thermo Fisher Scientific catalog PA5-18467), Inversin (Proteintech catalog 10585-1-AP), Dynein (Thermo Fisher Scientific catalog MA1-070), gTubulin (GeneTex catalog GTX113286), Alk1 (Abcam catalog ab51870), KLF4 (Proteintech catalog 11880-1-AP), HO-1 (BD, catalog 566391), NRF2 (BioLegend, catalog 939202), and bactin (Sigma catalog A5441 and Cell Signaling Technology catalog 4970P). Secondary antibodies used are goat anti-mouse PECy7 (BioLegend), donkey anti-rabbit PE (Thermo Fisher Scientific), donkey anti-goat AF657 (Thermo Fisher Scientific), donkey anti-rabbit BV421 (BioLegend), and donkey anti-rabbit AF488 (Thermo Fisher Scientific).

### Cell Culture

Primary HBMVECs (Cell Systems Corporation catalog ACBRI 376) and HUVECs (Glyco Tech) were maintained at 37° C. in a 5% CO₂ incubator in endothelial cell complete medium (Promocell, catalog C22010). As per vendor's description, cells were isolated from the cortex region of the brain from a pediatric male donor. The cells were isolated without using antibody labeling to preserve the cells' natural properties for enhanced biological relevance. LL-CPK1 (CL101.1TM) porcine renal epithelial cells from proximal tubule were obtained from ATCC. All cell culture wells were seeded equally, and wells were randomized to control versus experimental conditions with duplicates or triplicates per condition. All experiments were performed between passages 4 and 6. Cells that had reached approximately 90% confluence were used in the shear stress experiment. For ROS quantification experiments, cells were treated with PMA (Sigma, catalog P8139-5MG) at a concentration of 50 ng/mL for 1 hour. To inhibit ROS production in respective experimental groups, cells were treated with 20 μM NOX inhibitor VAS 2870 (Sigma, catalog SML0273-5MG) 1 hour prior to PMA treatment.

### Human Patient Studies

Blood samples were collected from adult patients with SCD homozygous for Hb S and from healthy adult donors. Patients and donors were recruited under the study protocol approved at Duke University. All patients with SCD had not been transfused for at least 3 months and had not experienced an acute vaso-occlusive crisis for the past 3 weeks, and 98% of the patients tested were on hydroxyurea. Blood samples were collected into citrate tubes. All RBCs were washed in PBS with Ca²⁺ and Mg²⁺ with collection of the plasma and removal of buffy coat.

### Western Blot

Plasma isolated from blood collected from patients with SCD (SS) and healthy (AA) volunteers was used for quantification of the following proteins: ARL13b, IFT88, and γ-tubulin. These samples were run on SDS-PAGE, and traditional Western blot protocols were performed. Primary antibodies used were explained before. Secondary antibodies used include anti-rabbit HRP (catalog 7074, Cell Signaling Technology) and anti-mouse HRP (catalog 7076, Cell Signaling Technology). Quantification was done using ImageJ software (NIH) and plotted against the housekeeping control protein bACTIN.

### In Vitro EC Shear Stress Experiments

Ibidi perfusion. To generate shear stress in vitro, we utilized the Ibidi pump system. Prior to initiating perfusion, HBMVECs were seeded onto a μ-slide (0.6 mm channel height; Ibidi, 81106) at 0.5×10⁶ cells per slide. Slides, media, and a perfusion set (red, 10962) were incubated in a humidified cell culture incubation chamber (37° C. in 5% CO₂) overnight prior to perfusion to prevent bubble formation, per manufacturer recommendations. Immediately prior to perfusion, medium was added to the syringe reservoirs (12 mL total) and air bubbles were removed. The μ-slide was attached to the perfusion set under sterile conditions, and then the fluidic unit was connected to the Ibidi pump and air tubing inside the cell culture incubation chamber. Using the vendor-specific PumpControl software, HBMVECs were perfused at 0, 2, 4, or 10 dyne/cm² for 10 minutes or 24 hours. A total of 2 dyne/cm² was included as representative of steady state that mimics in vivo condition. To prevent acute cell detachment upon initiation of high shear stress at 10 dyne/cm², cells were acclimatized to flow at 4 dyne/cm² for 5 minutes and then subjected to flow at 10 dyne/cm² for either 10 minutes or 24 hours. For experiments at 2 and 4 dyne/cm², no acclimatization was necessary, and cells were subjected to flow for 10 minutes or 24 hours. Per manufacturer recommendations with the 0.6 mm μ-slides and red perfusion sets, the minimum shear stress possible was 2.3 dyne/cm² (rounded off to and referred to as 2 dyne/cm²). Immediately following perfusion, cells were trypsinized with TrypLE express (Thermo Fisher Scientific, 12604013) and used for downstream analysis. All the samples subjected to any magnitude of perfusion had their respective no flow controls. Any protein expression in a given sample was normalized against that of no flow control.

### Shaker Method

A shaking incubator (New Brunswick Scientific) was used as previously described to induce shear stress (43). The formula used applied to calculate the stress is: shear stress=(6×F×m)/(w×h²), where F=moment of inertia (i.e., function of centrifugal force based on rpm and size of the shaker), m=viscosity of the fluid (i.e., function of temperature), w=diameter of plates (i.e., function of area of dish), and h=height of the fluid (i.e., function of volume).

A 100 mm culture dish (confluent with cells) that was placed on an orbital shaker at 240 rotations per minute (RPM) resulted in a shear stress of 10 dyne/cm² and at 96 RPM resulted in a shear stress of 4 dyne/cm². The cells were under shear stress for 4 minutes.

For HUVEC and epithelial cell shear stress experiments, the deciliation was induced by mechanical force, as previously described (43, 44). Cell populations were first rinsed briefly and gently with 10 mL of PBS (pH 7.4). A 150 mm culture dish was placed in a flow chamber as previously discussed (6). A shear stress of 10 or 20 dyne/cm² was applied to the cell for 4 minutes. The media containing the excised cilia were carefully transferred to a 50 mL centrifuge tube and centrifuged for 30 minutes at 3000 g at 4° C. The supernatant containing the excised cilia was then transferred to a polyallomer tube and centrifuged for 1 hour at 70,000 g at 4° C. in an ultracentrifuge. The purified primary cilia were then resuspended in the PBS buffer or RIPA buffer for further analyses.

### Quantitative Reverse-Transcription PCR

Total RNA was extracted using TRI Reagent and Direct-zol RNA Miniprep (Zymo Research, R2051). RNA was reverse-transcribed into cDNA using 250 ng total RNA (iScript gDNA clear cDNA synthesis kit; BioRad, 172-5034). RNA levels were quantified using custom-designed primers (Primer3) for KLF2, KLF4, and GAPDH. cDNA and primers were mixed with iTaq Universal SYBR Green Supermix (BioRad, 172-5121) and run with the following cycling protocol: 95° C. for 2:00 minutes, followed by 40 cycles of 95° C. for 0:10 minutes and 60° C. for 0:30 minutes (BioRad, CFX96 Real-Time System). Quantification of gene expression was performed using the 2-ΔΔCT method (45). Samples were all run in quadruplicate, and target genes were normalized to GAPDH.

Primers were KLF2 (133 bp)—forward: CACCAAGAGTTCGCATCTGA (SEQ ID NO: 1), reverse: CGTGTGCTTTCGGTAGTGG (SEQ ID NO: 2); KLF4 (132 bp)—forward: CGGCTGTGGATGGAAATTCG (SEQ ID NO:3), reverse: ATGTGTAAGGCGAGGTGGTC (SEQ ID NO: 4); and GAPDH(128 bp)—forward: CCAAGGAGTAAGACCCCTGG (SEQ ID NO: 5), reverse: CAACTGTGAGGAGGGGAGAT (SEQ ID NO: 6).

### In Vitro Adhesion Assays

HBMVECs were cultured until they reached confluence on clear glass slides precoated with 2% gelatin. Slides coated with brain ECs were washed, then fit into a variable height flow chamber and tested for their ability to support adhesion of RBCs. The flow chamber was mounted on the stage of an inverted phase contrast microscope (Diaphot, Nikon Inc.) connected to a thermoplate (Tokai Hit Co., Ltd.) set at 37° C. Fluorescence-labeled RBCs suspended at 0.2% (v/v) in PBS with Ca²⁺ and Mg²⁺ were infused into the flow chamber and allowed to adhere to brain ECs for 10 minutes without flow. Before exposure to flow, a minimum of 3 fields at each of 7 different locations along a line oriented to future flow were examined, and the total number of fluorescent cells was counted. Fluid flow (PBS with Ca²⁺ and Mg²⁺) with a calibrated syringe pump was then started for a period of 15 minutes. Effluent was collected and tested by flow cytometry for cilia shedding from brain ECs by RBC contact. After exposure to flow, the fields were examined, and the number of fluorescent adherent RBCs to brain ECs was counted. The fraction of adherent cells was presented as number of cells attached per field after exposure to flow/total number of cells present per field before flow.

The wall shear stress was calculated as t_{w}=6Qm/wh², where t_{w}=wall shear stress (dyne/cm²); Q=volumetric flow rate (cm³/s); m=media viscosity, w=width of the flow channel, and h=height of the flow chamber as a function of position along the microscope slide. Blood flow in small vessels may be continuous (nonpulsatile) with shear stresses of 1-2 dyne/cm², or flow may be intermittent (pulsatile). Our data were obtained using pulsatile flow conditions.

### Shear Stress Zebrafish Flow Parameter Measurements

The transgenic line Tg(bact:Arl13b-GFP) was obtained from Brian Ciruna (University of Toronto, Toronto, Ontario, Canada). Tg(kdrl:mCherry-CAAX), Casper, and wild-type AB lines were obtained from Zebrafish Informational Resource Center. Embryos from the wild-type (AB) strain were used in this study. Freshly fertilized embryos were procured through natural breeding of adult zebrafish and were raised at 28.0° C. in E3 medium containing 0.1 mM N-Phenylthiourea (PTU; Sigma) to inhibit pigment formation. For shear stress experiments, at 28 hpf, fish embryos were divided into 3 groups: G1: 0-48 hpf (28° C.); G2: 0-28 hpf (28° C.) followed by 3.5 or 5 hours of incubation at 32° C. or 35° C. and returning to 28° C. until 48 hpf, and G3: 0-28 hpf (28° C.) followed by incubation at 32° C. or 35° C. until 48 hpf (FIG. 10). Control and experimental embryos were subsequently dechorionated at 48 hpf for imaging. A stereomicroscope (Zeiss SteREO Discovery V12 Microscope equipped with Hamamatsu Orca Flash high-speed camera and a workstation equipped with HCImage software, Hamamatsu Photonics) was used to visualize the blood vessels of zebrafish embryos as previously described (46). High-speed video microscopy movies of the heart and tail in 1000 frames per 10 seconds at 100× magnification were recorded for the PMBCs and the DA. The recorded movies were analyzed according to our previous protocols (47, 48) using MicroZebraLab blood flow from Viewpoint (version 3.4.4), and 4 cardiac parameters were calculated for each vessel: pulse, blood flow velocity, vessel diameter, and shear stress using the following formula: τ=4 μVₘₑₐₙ/D, where μ=blood viscosity (dyne/cm²), V=average blood velocity (μm/s), and D=vessel diameter (μm).

This experiment was performed multiple times, and data from 3 independent experiments are reported.

### Whole-Mount Staining of Casper Zebrafish Using O-Dianisidine

Embryos from Casper strain were used to visualize hemorrhage in the brain after the induction of shear stress as described in the section above. At 48 hpf, embryos were dechorionated and stained with O-dianisidine (stains RBCs). The stain was prepared by mixing 0.6 mg/mL of O-dianisidine (Sigma), 0.65% hydrogen peroxide, 0.01 M sodium acetate (pH 4.5), and 40% (v/v) ethanol solution. Embryos were washed with PTU-E3 medium, which is N-Phenylthiourea dissolved in egg water made in-house, and then 0.6 mg/mL of the stain was added and left for 15 minutes in the dark. After staining, embryos were postfixed in 4% paraformaldehyde at 4° C. for at least 1 hour. Three percent (w/v) methyl cellulose was used to fix the embryos on the slide for imaging under a bright-field microscope (Stemi 508, Zeiss). Zeiss AxioCam ERc 5s professional digital camera was used for imaging.

### In Vivo (Zebrafish) Shear Stress Experiments

Embryos from a cross of Tg(bact:Arl13b-GFP) and Tg(kdrl:mCherry-CAAX) were used in this study (FIG. 9B). Freshly fertilized embryos were procured through natural breeding of adult zebrafish and were kept at 28.0° C. in 1×E3 embryo medium (E3 medium) containing 5 mmol/L NaCl, 0.17 mmol/L KCl, 0.33 mmol/L CaCl₂), 0.33 mmol/L MgSO₄, and 0.05% methylene blue. For shear stress experiments, at 29.5 hpf, fish embryos were transferred to an incubator at 32.0° C. for 3 hours. Control and experimental embryos were subsequently dechorionated and digested to yield single cells (FIG. 9B). The composition of digestion buffer used was RPMI 1640 medium (Thermo Fisher Scientific) supplemented with 10% FCS, collagenase D (1 mg/mL), and DNase I (10 μg/mL). Embryos were digested for 30 minutes at 37° C. and subsequently passed through a 70 μm cell strainer. Cells were centrifuged at 300 g for 5 minutes and washed twice with PBS before use for downstream applications. This experiment was performed multiple times, and data from 3 independent experiments are reported.

### Flow Cytometry

Single-cell suspensions were washed 3 times with FACS buffer (1×PBS with 5% FBS and 0.1% NaN₃) at 300 g for 5 minutes and were subsequently incubated with Live/Dead fixable yellow dead cell stain as per manufacturer's protocol, to exclude any dead cells, wherever applicable. Then, cells were fixed and permeabilized using Cytofix/Cytoperm buffer (BD, catalog 554722) or transcription factor buffer set (BD, catalog 562574) and stained for the following proteins: ARL13b, IFT88, Inversin, Dynein, γ-tubulin, Alk1, KLF4, HO-1, NRF2, and bACTIN. Suitable secondary reagents were used to detect the respective proteins. Primary antibodies were diluted 1:50, and secondary antibodies were diluted 1:500. BD perm wash buffer (catalog 554723) was used for antibody dilutions and washing. Primary antibodies were incubated for 45 minutes and secondaries for 30 minutes at 4° C. Suitable secondary antibody controls were included. To quantify total ROS, assay kit was used as per the manufacturer's instruction (Thermo Fisher Scientific, catalog 88-5930-74). After the completion of staining, cells were resuspended in FACS buffer. Stained cells were run on a flow cytometer (BD LSRFortessa). Sample acquisition was done using FACSDiva software (BD) with subsequent analysis on FlowJo software. To determine the presence of the ciliary protein ARL13b on human RBCs, unlabeled RBCs prior to infusion into the variable height flow chamber, and effluent containing RBCs collected postflow and RBCs postinteraction with brain ECs, were labeled with FITC-conjugated Arl13b antibody for 30 minutes on ice. RBCs were then washed and tested by flow cytometric analysis as previously described (49). To determine whether ROS generation in brain ECs can be increased by sickle RBCs and contributes to deciliation, slides coated with brain ECs were sham-treated or pretreated with the NOX inhibitor apocynin at 10 μM for 1 hour at 37° C., washed, and fitted into the flow chamber. Treated brain ECs were then exposed to unlabeled sickle RBCs for 10 minutes. Sickle RBCs were lysed with RBC lysis buffer and brain ECs scraped from the glass slide for testing for ROS levels using CM-H2-DCFDA (Invitrogen) as described previously in detail (50). One hundred thousand events per sample were acquired and tested by flow cytometric analysis. In separate experiments, and as described above, unlabeled sickle RBCs were tested by flow cytometry for ARL13b ciliary protein binding prior to infusion into the chamber (baseline levels) and postflow and once sickle RBCs interacted with brain ECs treated with 10 μM apocynin.

### Mouse Sickle RBCs: Cilia Staining and Quantification

For the blood smear preparation, approximately 5 μL of whole blood from AA control mouse and SS sickle mouse was added to a glass slide and allowed to dry for 24 hours. The smear was fixed with acetone for 10 minutes. At the end of incubation, the slides were washed with PBS, and the primary antibody (ARL13b or IFT88) at the concentration of 1:500 was added to the slides and incubated at 4° C. overnight. The slides were washed with PBS, and secondary antibody was added at a concentration of 1:500 and incubated for 1 hour at room temperature in the dark. At the end of incubation, the slides were washed with PBS, the mounting reagent was added, and the coverslip was placed on top of the blood smear and imaged at 63× on a confocal microscope (Zeiss LSM 510 laser module) with bright-field for RBCs and 488 green channel for ARL13b- or IFT88-positive cilia detection. All the cilia-positive RBCs were quantified with multipoint tool in ImageJ software and represented as graphs.

### Statistics

Data were presented as mean and SEM. A 2-tailed t test or 1- or 2-way ANOVA was performed to compare groups on the outcomes. Pearson's correlation and regression analysis were used to investigate the relationships between continuous variables. Cilia protein in vivo under different sheer stress was expressed as fold change relative to the mean of the control group. A linear mixed model (LMM) was then used to examine differences between experimental and control groups. The differences of cilia proteins (ARL13b, γ-tubulin, IFT88, and Inversin) within EC (mCherry⁺) or non-EC (mCherry) were also analyzed by LMM. P<0.05 was considered significant. Dunnett's test, Tukey's test, or Bonferroni correction was used to adjust for multiple comparisons. Data were log-transformed to improve fit for some analyses. Nonparametric tests were used where parametric assumptions were not satisfied. Statistical analysis was performed using SAS V9.4 (SAS Institute Inc.), R, and GraphPad Prism software (version 9.0).

### REFERENCES

1. Malicki J J, Johnson C A. The cilium: cellular antenna and central processing unit. Trends Cell Biol. 2017; 27(2):126-140.
2. Goetz S C, Anderson K V. The primary cilium: a signalling centre during vertebrate development. Nat Rev Genet. 2010; 11(5):331-344.
3. Nguyen Q P H, et al. Comparative super-resolution mapping of basal feet reveals a modular but distinct architecture in primary and motile cilia. Dev Cell. 2020; 55(2):209-223.
4. Egorova A D, et al. Primary cilia as biomechanical sensors in regulating endothelial function. Differentiation. 2012;83(2):S56-S61.
5. Hierck B P, et al. Primary cilia sensitize endothelial cells for fluid shear stress. Dev Dyn. 2008; 237(3):725-735.
6. Nauli S M, et al. Endothelial cilia are fluid shear sensors that regulate calcium signaling and nitric oxide production through polycystin-1. Circulation. 2008; 117(9):1161-1171.
7. AbouAlaiwi W A, et al. Ciliary polycystin-2 is a mechanosensitive calcium channel involved in nitric oxide signaling cascades. Circ Res. 2009; 104(7):860-869.
8. Mohieldin A M, et al. Vascular endothelial primary cilia: mechanosensation and hypertension. Curr Hypertens Rev. 2016; 12(1):57-67.
9. Pala R, et al. The roles of primary cilia in cardiovascular diseases. Cells. 2018;7(12):E233.
10. Luu V Z, et al. Role of endothelial primary cilia as fluid mechanosensors on vascular health. Atherosclerosis. 2018; 275:196-204.
11. Goetz J G, et al. Endothelial cilia mediate low flow sensing during zebrafish vascular development. Cell Rep. 2014; 6(5):799-808.
12. Abdul-Majeed S, et al. Mechanisms regulating cilia growth and cilia function in endothelial cells. Cell Mol Life Sci. 2012; 69(1):165-173.
13. Iomini C, et al. Primary cilia of human endothelial cells disassemble under laminar shear stress. J Cell Biol. 2004; 164(6):811-817.
14. Dinsmore C, Reiter J F. Endothelial primary cilia inhibit atherosclerosis. EMBO Rep. 2016; 17(2):156-166.
15. Vion A C, et al. Primary cilia sensitize endothelial cells to BMP and prevent excessive vascular regression. J Cell Biol. 2018; 217(5):1651-1665.
16. Eisa-Beygi S, et al. Characterization of endothelial cilia distribution during cerebral-vascular development in zebrafish (Danio rerio). Arterioscler Thromb Vasc Biol. 2018; 38(12):2806-2818.
17. Mirvis M, et al. Primary cilium loss in mammalian cells occurs predominantly by whole-cilium shedding. PLoSBiol. 2019;17(7):e3000381.
18. Kong M J, et al. Fragmentation of kidney epithelial cell primary cilia occurs by cisplatin and these cilia fragments are excreted into the urine. Redox Biol. 2019; 20:38-45.
19. Kallakuri S, et al. Endothelial cilia are essential for developmental vascular integrity in zebrafish. JAm Soc Nephrol. 2015; 26(4):864-875.
20. Arnold C R, et al. Comparative analysis of genes regulated by Dzipl/iguana and hedgehog in zebrafish. Dev Dyn. 2015; 244(2):211-223.
21. Lamont R E, et al. Hedgehog signaling via angiopoietinl is required for developmental vascular stability. Mech Dev. 2010;127(3-4):159-168.
22. Jones T J, et al. Primary cilia regulates the directional migration and barrier integrity of endothelial cells through the modulation of hsp27 dependent actin cytoskeletal organization. J Cell Physiol. 2012; 227(1):70-76.
23. Ma N, Zhou J. Functions of endothelial cilia in the regulation of vascular barriers. Front Cell Dev Biol. 2020; 8:626.
24. Cortellino S, et al. Defective ciliogenesis, embryonic lethality and severe impairment of the Sonic Hedgehog pathway caused by inactivation of the mouse complex A intraflagellar transport gene Ift122/Wdr10, partially overlapping with the DNA repair gene Med1/Mbd4. Dev Biol. 2009; 325(1):225-237.
25. Gorivodsky M, et al. Intraflagellar transport protein 172 is essential for primary cilia formation and plays a vital role in patterning the mammalian brain. Dev Biol. 2009; 325(1):24-32.
26. DeStefano J G, et al. Effect of shear stress on iPSC-derived human brain microvascular endothelial cells (dhBMECs). Fluids Barriers CNS. 2017;14(1):20.
27. Worthen L M, Nollert M U. Intracellular calcium response of endothelial cells exposed to flow in the presence of thrombin or histamine. J Vasc Surg. 2000; 32(3):593-601.
28. Zhang F, et al. Effects of fluid shear stress on expression of Smac/DIABLO in human umbilical vein endothelial cells. Curr Ther Res Clin Exp. 2013; 74:36-40.
29. Barrionuevo W R, Burggren W W. 02 consumption and heart rate in developing zebrafish (Danio rerio): influence of temperature and ambient 02. Am J Physiol. 1999;276(2 pt 2):R505-R513.
30. Baker K, et al. Defective “pacemaker” current (Ih) in a zebrafish mutant with a slow heart rate. Proc NatlAcad Sci USA. 1997; 94(9):4554-4559.
31. Ingram V M. A specific chemical difference between the globins of normal human and sickle-cell anaemia haemoglobin. Nature. 1956; 178(4537):792-794.
32. Switzer J A, et al. Pathophysiology and treatment of stroke in sickle-cell disease: present and future. Lancet Neurol. 2006; 5(6):501-512.
33. Zennadi R, et al. Sickle red cells induce adhesion of lymphocytes and monocytes to endothelium. Blood. 2008; 112(8):3474-3483.
34. Zennadi R, et al. Epinephrine acts through erythroid signaling pathways to activate sickle cell adhesion to endothelium via LW-alphavbeta3 interactions. Blood. 2004; 104(12):3774-3781.
35. Sultana C, et al. Interaction of sickle erythrocytes with endothelial cells in the presence of endothelial cell conditioned medium induces oxidant stress leading to transendothelial migration of monocytes. Blood. 1998; 92(10):3924-3935.
36. Reneman R S, Hoeks A P. Wall shear stress as measured in vivo: consequences for the design of the arterial system. Med Biol Eng Comput. 2008; 46(5):499-507.
37. Nader E, et al. The red blood cell-inflammation vicious circle in sickle cell disease. Front Immunol. 2020; 11:454.
38. Stuart J, Johnson C S. Rheology of the sickle cell disorders. Baillieres C/in Haematol. 1987; 1(3):747-775.
39. Kurantsin-Mills J, et al. Flow dynamics of human sickle erythrocytes in the mesenteric microcirculation of the exchange-transfused rat. Microvasc Res. 1987; 34(2):152-167.
40. Kim J I, et al. Reduction of oxidative stress during recovery accelerates normalization of primary cilia length that is altered after ischemic injury in murine kidneys. Am JPhysiol Renal Physiol. 2013;304(10):F1283-F1294.
41. Erdbruegger U, et al. Circulating endothelial cells: markers and mediators of vascular damage. Curr Stem Cell Res Ther. 2010; 5(4):294-302.
42. Tenreiro M M, et al. Cellular response of the blood-brain barrier to injury: potential biomarkers and therapeutic targets for brain regeneration. Neurobiol Dis. 2016; 91:262-273.
43. Mohieldin A M, et al. Protein composition and movements of membrane swellings associated with primary cilia. Cell Mol Life Sci. 2015; 72(12):2415-2429.
44. Mohieldin A M, et al. Proteomic identification reveals the role of ciliary extracellular-like vesicle in cardiovascular function. Adv Sci (Weinh). 2020;7(16):1903140.
45. Schmittgen T D, Livak K J. Analyzing real-time PCR data by the comparative C(T) method. Nat Protoc. 2008; 3(6):1101-1108.
46. Al-Kandari H, et al. Ecotoxicological assessment of thermally- and hydrogen-reduced graphene Oxide/TiO2 photocatalytic nanocomposites using the zebrafish embryo model. Nanomaterials (Basel). 2019;9(4):488.
47. Benslimane F M, et al. Cardiac function and blood flow hemodynamics assessment of zebrafish (Danio rerio) using high-speed video microscopy. Micron. 2020; 136:102876.
48. Yalcin H C. Hemodynamic Studies for Analyzing the Teratogenic Effects of Drugs in the Zebrafish Embryo. In: Felix L, ed. Teratogenicity Testing: Methods and Protocols. Springer; 2018:487-495.
49. Udani M, et al. Basal cell adhesion molecule/lutheran protein. The receptor critical for sickle cell adhesion to laminin. J Clin Invest. 1998; 101(11):2550-2558.
50. MacKinney A, et al. Disrupting the vicious cycle created by NOX activation in sickle erythrocytes exposed to hypoxia/reoxygenation prevents adhesion and vasoocclusion. Redox Biol. 2019; 25:101097.

We claim:

1.-29. (canceled)

30. A method of sample processing, comprising:
(a) analyzing cells of a blood sample obtained from a subject, comprising:
(i) selectively identifying red blood cells of the blood sample; and
(ii) detecting at least one cilia biomarker associated with the red blood cells of the blood sample; and
(b) quantifying the at least one cilia biomarker associated with the red blood cells of the blood sample.

31. The method of claim 30, wherein step (a)(i) comprises flow cytometry.

32. The method of claim 30, further comprising comparing (i) a quantity of the at least one cilia biomarker associated with the red blood cells of the blood sample resulting from the quantifying in (b), and (ii) a control quantity of the at least one cilia biomarker associated with one or more control red blood cells in one or more control subjects.

33. The method of claim 32, wherein the quantifying the at least one cilia biomarker in (b) indicates a presence of, or a risk of developing, a brain injury in the subject, when the quantity of the at least one cilia biomarker is higher than the control quantity of the at least one cilia biomarker associated with the one or more control red blood cells in the one or more control subjects, wherein the one or more control subjects does not have the brain injury.

34. The method of claim 32, wherein the quantifying the at least one cilia biomarker in (b) indicates a presence of, or a risk of developing, a condition, when the quantity of the at least one cilia biomarker is higher than the control quantity of the at least one cilia biomarker associated with the one or more control red blood cells in the one or more control subjects, wherein the one or more control subjects does not have the condition, wherein the condition comprises sickle cell disease, preeclampsia, polycystic kidney disease, stroke or pulmonary hypertension.

35. The method of claim 30, further comprising performing (a)-(b) on a second blood sample obtained from the subject at an earlier time point than the blood sample.

36. The method of claim 30, wherein the detecting the at least one cilia biomarker in (a)(ii) comprises:
(1) contacting the blood sample with at least one antibody that binds to the at least one cilia biomarker, wherein the at least one antibody is labeled; and
(2) measuring a quantity of the at least one antibody.

37. The method of claim 30, wherein the at least one cilia biomarker comprises ADP-ribosylation factor-like protein 13B (ARL13b), intraflagellar transport 88 (IFT88) protein, or a combination thereof.

38. The method of claim 30, further comprising determining a relative risk of the subject developing vascular damage in brain tissue based, at least in part, on the quantifying the at least one cilia biomarker in (b) relative to a control or index derived from one or more control subjects that does not have vascular damage in brain tissue.

39. A method of processing a sample, comprising:
(a) producing a fraction of a blood sample obtained from a subject, comprising:
(i) introducing the blood sample to an antibody under conditions sufficient to bind the antibody to one or more cilia biomarkers in the blood sample, wherein the antibody is immobilized to a solid support; and
(ii) selectively removing components of the blood sample that are not bound to the antibody; and
(b) quantifying the at least one cilia biomarker within the fraction, wherein the at least one cilia biomarker is associated with a red blood cell in the blood sample.

40. The method of claim 39, comprising performing (a)-(b) using an enzyme-linked immunoassay (ELISA).

41. The method of claim 39, further comprising processing the blood sample before (a), wherein the processing comprises lysing the blood sample.

42. The method of claim 39, wherein a quantity of the at least one cilia biomarker resulting from the quantifying in (b) indicates a presence or an absence of a condition.

43. The method of claim 42, wherein the condition comprises brain injury, sickle cell disease, preeclampsia, polycystic kidney disease, stroke, or pulmonary hypertension.

44. The method of claim 39, further comprising performing (a)-(b) on a second blood sample.

45. The method of claim 44, wherein the second blood sample is obtained from the subject at an earlier time point than the blood sample.

46. The method of claim 44, wherein the second blood sample is from a second subject, wherein the second subject does not have a condition selected from the group consisting of brain injury, sickle cell disease, preeclampsia, polycystic kidney disease, stroke, and pulmonary hypertension.

47. The method of claim 39, wherein the at least one cilia biomarker comprises ADP-ribosylation factor-like protein 13B (ARL13b), intraflagellar transport 88 (IFT88) protein, or a combination thereof.

48. The method of claim 39, wherein the at least one cilia biomarker comprises inversin protein, acetylated-a-tubulin, Dynein, γ-tubulin protein, or nuclear factor-erythroid factor 2-related factor 2 (NRF2) protein, or a combination thereof.

49. The method of claim 39, further comprising providing a relative risk of the subject developing vascular damage in brain tissue based at least in part on the quantifying in (b).


SEQUENCE LISTING (6 sequences)

SEQ ID NO: 1 - DNA, 20 bp, Artificial Sequence
  Synthetic- KLF2 forward primer
caccaagagttcgcatctga

SEQ ID NO: 2 - DNA, 19 bp, Artificial Sequence
  Synthetic- KLF2 reverse primer
cgtgtgctttcggtagtgg

SEQ ID NO: 3 - DNA, 20 bp, Artificial Sequence
  Synthetic- KLF4 forward primer
cggctgtggatggaaattcg

SEQ ID NO: 4 - DNA, 20 bp, Artificial Sequence
  Synthetic- KLF4 reverse primer
atgtgtaaggcgaggtggtc

SEQ ID NO: 5 - DNA, 20 bp, Artificial Sequence
  Synthetic- GAPDH forward primer
ccaaggagtaagacccctgg

SEQ ID NO: 6 - DNA, 20 bp, Artificial Sequence
  Synthetic- GAPDH reverse primer
caactgtgaggaggggagat